(19) United States
(12) Patent Application Publication
(10) Pub. No.: US 2025/0032617 A1
(43) Pub. Date: Jan. 30, 2025

(54) TRANSGENIC T CELL RECEPTORS TARGETING NEOANTIGENS FOR DIAGNOSIS, PREVENTION, AND/OR TREATMENT OF HEMATOLOGICAL CANCERS

(71) Applicant: Baylor College of Medicine, Houston, TX (US)

(72) Inventors: Wingchi Leung, Houston, TX (US); Premal Lulla, Houston, TX (US); Ann Marie Leen, Houston, TX (US); Valentina Hoyos, Houston, TX (US); Paul Shafer, Houston, TX (US)

(73) Assignee: Baylor College of Medicine, Houston, TX (US)

(21) Appl. No.: 18/698,685

(22) PCT Filed: Oct. 7, 2022
(86) PCT No.: PCT/US2022/077729

§ 371 Date: Apr. 4, 2024

Related U.S. Application Data
(60) Provisional application No. 63/253,741, filed on Oct. 8, 2021.

(51) Int. Cl. A61K 39/00; C12N 5/0783; G01N 33/50

(52) U.S. Cl. CPC A61K 39/464499; A61K 39/4611; C12N 5/0636; G01N 33/5011; G01N 33/505; C12N 2501/22; C12N 2501/2301; C12N 2501/2304; C12N 2501/2306; C12N 2501/2307; C12N 2501/2312; C12N 2501/2315; C12N 2501/25; C12N 2502/30

(57) ABSTRACT

Embodiments of the disclosure include methods and compositions for producing T cell receptor (TCR) polypeptides specific for hematological neoantigens. In specific embodiments. T cells directed to one or more hematological neoantigens are produced following exposure of PBMCs to peptides that encompass one or more neoantigens, and the TCRs in the produced T cells are tested for efficacy and identified. The neoantigen-specific TCRs are utilized in a variety of immunotherapies.

This application claims priority to U.S. Provisional Patent Application Ser. No. 63/253,741, filed Oct. 8, 2021, which is incorporated by reference herein in its entirety.

### STATEMENT REGARDING FEDERALLY SPONSORED RESEARCH OR DEVELOPMENT

This invention was made with government support under P50 CA126752 awarded by National Institutes of Health-National Cancer Institute. The government has certain rights in the invention.

### TECHNICAL FIELD

Embodiments of the present disclosure generally concern at least the fields of cell biology, immunology, molecular biology, and medicine. In particular embodiments, the disclosure generally concerns immunotherapy for cancer.

### BACKGROUND

While adoptively transferred chimeric antigen receptor (CAR)-modified T cells have transformed the treatment of CD19+ hematological malignancies, successful extension to other haematological cancers, such as AML has been hindered by the lack of tumor-exclusive target antigens. For example, surface-expressed CD33 and CD123 are also present on myeloid precursors whose ablation is clinically intolerable. Fortunately, leukemic blasts have a rich pool of intracellular antigens (called neoantigens), which arise from non-synonymous mutations within malignant cells and can theoretically be targeted by T cells via their native T cell receptor (TCR), while sparing healthy tissues. The present disclosure describes the methods of generating transgenic TCRs targeting neoantigens in hematological maliganancie that arise from mutations-including 1) driver mutations which drive the malignant phenotype, and/or 2) “hot spot” mutations that are frequently detected within specific tumors or shared between tumor types. Such mutations can arise in genes such as DNMT3A, IDH1, IDH2 and NPM1, JAK, FLT3, KRAS and NRAS. The present disclosure also describes the application of transgenic TCRs for the diagnosis, prevention and treatment and treatment of hematological malignancies.

The present disclosure satisfies a long-felt need in the art to provide effective cancer-specific cell immunotherapies against hematological cancers.

### BRIEF SUMMARY

The present disclosure relates to the production of transgenic TCRs that target one or more neoantigens associated with hematological cancers. In specific embodiments, these TCRs can be utilized as adoptive cell therapy or other TCR-based techniques for the detection, prevention, and/or treatment of one or more hematological cancers.

In specific embodiments, the present disclosure provides methods to produce immunotherapies of any kind utilizing transgenic TCRs that target one or multiple neoantigens associated with hematological cancers or precancerous conditions. In specific embodiments, a neoantigen associated with hematological cancers or precancerous conditions is a neoantigen that is respectively present in at least some hematological tumor cells of the individual or in at least some precancerous cells (cells that carry a mutation leading to a precancerous condition including clonal hematopoiesis, myelodysplastic syndromes, monoclonal B lymphocytosis, monoclonal gammopathy) of the individual. In specific embodiments, the hematological neoantigen comprises one or more mutations of any kind.

In specific embodiments, the source of the hematological neoantigen used to produce the neontigen-targeted T cells may comprise peptides encompassing the mutated sequence, used either alone or pooled with peptides encompassing a mutated sequence of another neoantigen, such as to target multiple neoantigens. The source of the neoantigen comprising peptides may be a library of peptides, in which each in the library that are identical, or the library may comprise a plurality of different peptides, though some peptides in the library may be duplicative in sequence of others.

In certain embodiments, the neoantigen-specific T cells are cultured under specific conditions and/or environments, such as being cultured in tissue cultureware including multiwell substrates (e.g., 24-well tissue culture treated plates) and/or in vessels, such as vessels having gas permeable membranes. In specific embodiments, the culture medium comprises one or more specific cytokines, and the culturing may occur using one stimulation or multiple stimulations. When there are multiple stimulations, the specific conditions and/or environments may or may not be the same among the stimulations. For example, one or more subsequent stimulations following an initial stimulation may use a different tissue cultureware and/or different culture medium. In some cases, one or more subsequent stimulations following an initial stimulation comprises a different combination of cytokines, and one or more cytokines in the different stimulations may or may not overlap. In a specific case, the culture medium comprises IL-7, IL-12, IL-15, and IL-6 during an initial stimulation and comprises IL-7 and IL-15 and/or IL-2 at a subsequent stimulation, such as a second and/or further stimulation. In specific embodiments, the culture medium comprises 1, 2, 3, 4, 5, or more cytokines, and in other embodiments the culture medium consists essentially of 1, 2, 3, 4, or 5 or more cytokines, and in other embodiments, the culture medium consists of 1, 2, 3, 4, or 5 or more cytokines. In specific embodiments with respect to cytokines only, the culture medium comprises 1, 2, 3, 4, 5, or more cytokines, and in other embodiments the culture medium consists essentially of 1, 2, 3, 4, or 5 or more cytokines, and in other embodiments, the culture medium consists of 1, 2, 3, 4, or 5 or more cytokines; in such cases, the culture medium comprises one or more other components that are effective for cell stimulation and production.

For the generation of the neoantigen-specific T cells, antigen presenting cells (APCs) such as dendritic cells (DCs) may be pulsed with one or more peptides, including one or more libraries of peptides, encompassing a mutated neoantigen peptide sequence. Alternatively, PBMCs can be stimulated directly with one or more peptides, including one or more libraries of peptides, encompassing a mutated neoantigen sequence.

In some embodiments, this disclosure provides for the targeting of cancers (hematological and/or solid tumors) or precancerous conditions (such as clonal hematopoiesis of indeterminate potential) that harbor the target neo-antigen(s) using T cells stimulated with peptides encompassing mutated sequences present in tumors including driver mutations (i.e., mutations that drive the malignant phenotype; “hot spot” mutations (i.e., mutations that are frequently detected across many different diseases/tumor types); and/or patient-specific neo-peptides identified by sequencing strategies including whole genome sequencing (WGS) studies performed on fresh or banked tumor material.

The disclosure concerns, at least in part, protocols to generate T cell lines either from individuals with cancer or healthy donors. These T cell lines with specificity for one or multiple hematological neoantigens simultaneously may be generated using antigen presenting cells (e.g., DCs) pulsed with a mix of peptides encompassing mutant sequences. This approach allows for priming both CD4+ and CD8+ tumor-specific T cells. Targeting one or more neoantigens associated with hematological cancer or a precancerous condition maximizes the efficacy of adoptively transferred T cells and minimizes both the risk of off-tumor toxicities and the risk of tumor immune escape, in at least some embodiments. For in vitro expansion, in specific embodiments one can utilize an optimized cytokine cocktail to promote the selective expansion of tumor-reactive neospecific T cells. Resultant lines are tested for functionality in vitro such as by the production of one or multiple effector molecules including IL-2, IFNγ, TNFα and Granzyme B, for example when stimulated with neopeptides in ELIspot, Flurospot and intracellular cytokine assays, and specifically lysed peptide-pulsed target cells in traditional Cr51 release assays.

In particular embodiments, the TCRs from the produced neoantigen-specific T cells are identified, including sequenced, in at least some cases. Methods to identify TCRs include stimulating cells with peptides, and identifying the population of neoantigen-reactive cells (as determined by, e.g., production of one or more multiple effector molecules such as IL-2, IFNγ, TNFα, Granzyme B) or activation markers (such as CD137). The TCRs of reactive (neo-specific) cells are then identified using techniques such as VB repertoire staining by flow cytometry, DNA sequencing and/or RNA sequencing. For single cell analysis, the population of reactive (neo-specific) cells can also be first isolated (using, e.g., flow cytometery, magnetic bead selection), undergo limiting dilution and expansion prior to sequencing. The TCRs may be utilized in other cells, including any type of immune cells and including T cells, NK cells, NK T cells, macrophages, effector cells of any kind, and so forth. Cells that express the TCR from the produced neoantigen-specific T cells may be further modified, such as having disruption of one or more endogenous genes. TCRs can also be used in other forms of TCR-based immunotherapy, such as through conjugation with CD3 or drugs, including small molecules. In these cases, TCRs can be in its entirety, or limited to the regions encompassing the antigen-recognition domain).

In some cases, the TCRs from the produced neoantigen-specific T cells are utilized in diagnostic or detection embodiments, such as for detecting the neoantigen in a research or clinical sample. In this case, the presence of neoantigen-specific TCR sequences identified in an individual's memory T cells indicate the presence of pre-cancer or cancer.

In embodiments of the disclosure, there are methods of generating transgenic neoantigen-specific T cell receptor (TCR)-based immunotherapy that target at least one peptide comprising part or all of a neoantigen present in a hematological malignancy or pre-cancerous state, said method comprising the steps of: (a) contacting a population of antigen presenting cells (APCs) with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, thereby producing pepmix-loaded APCs; contacting peripheral blood mononuclear cells (PBMCs) from an individual with the pepmix-loaded APCs and performing at least one in vitro stimulation to produce a population of antigen-specific T cells that are capable of responding to at least one of the peptides; or (b) contacting PBMCs from an individual with cancer or a healthy individual with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, and performing at least one in vitro stimulation to produce a population of antigen-specific T cells that are capable of responding to at least one of the peptides; wherein in (a) or (b) the in vitro stimulation comprises culturing in a medium supplemented with two or more cytokines selected from the group consisting of IL-7, IL-12, IL-15 and IL-6 to produce the hematological neoantigen-specific T cells; and (c) generating the TCR-modified immunotherapy from the antigen binding region of the TCRs or the entire TCRs of the hematological neoantigen-specific T cells. In specific embodiments, the TCR-modified immunotherapy comprises TCR-modified immune cells, a TCR-drug conjugate, TCR-radiotherapy conjugate, TCR-linked CD3 complex, or a mixture thereof. The method may further comprise the step of assaying for neo-epitope specificity of the neoantigen-specific T cells by assessing T cell activity against one or more particular peptides comprising the mutated neoantigen sequence in comparison to T cell activity against a correlative peptide comprising the corresponding wild type sequence. The T cell activity may be assessed by production of IFNγ, TNFα and/or Granzyme B and/or by direct cytotoxicity of neopeptide-expressing targets. The T cell activity may be assessed by ELIspot, Flurospot, single cell RNA sequencing, cytotoxicity assay, and/or one or more intracellular cytokine assays.

Some embodiments of the method may further comprise the step of one or more additional in vitro stimulation steps. In specific embodiments, the one or more additional in vitro stimulation steps comprises culturing the cells in a medium comprising IL-7 and one or both of IL-15 and IL-2. The stimulation step may occur in a multiwell substrate or flask or a vessel with a gas permeable membrane. The APCs may be dendritic cells, allogeneic feeder cells, cells from lymphblastoid cell lines, activated T cells, PHA blasts with irradiation, PHA blasts without irradiation, B cells, monocytes, genetically modified mesenchymal stem cells, tumor cell lines, cells from K562 cell line modified to express one or more costimulatory molecules, a combination of stimulator cells that either present antigens and/or provide costimulation and/or produce one or more soluble factors that promote enrichment of the cells in vitro, or a mixture thereof. In some cases, the mutated neoantigen sequence comprises one or more modified amino acids, such as one or more amino acid substitutions, one or more amino acid deletions, one or more insertions, one or more inversions, or one or more translocations.

In particular embodiments, the PBMCs are from an individual that has cancer or a precancerous condition harboring one or more of the neoantigens. The PBMCs may be from an individual that is healthy and does not have cancer or a precancerous condition. In specific embodiments, the PBMCs from which the engineered hematological neoantigen-specific immune cells were produced are from an individual in need of therapy. In some embodiments, the PBMCs from which the engineered hematological neoantigen-specific immune cells were produced are not from the individual in need of therapy.

Some methods may further comprise the step of identifying the α and/or β T-cell receptor (TCR) polypeptide sequences of the neoantigen-specific T cells, such as by single cell sequencing methods or bulk sequencing methods. In some cases, specific neoantigen-reactive cells are captured using flow cytometry or magnetic selection for sequencing. Specific cells may be determined by IFNγ-secretion, TNFα secretion, or expression of activation markers such as CD137, CD28, and/or CD69. In some embodiments, the method further comprises the step of cloning the α and/or β T-cell receptor (TCR) sequences into a vector, wherein the α and/or β TCR sequences encompass the antigen binding domains or the entire TCR sequences. The vector may be a viral vector (retroviral, lentiviral, adenoviral, or adeno-associated viral vector) or a non-viral vector (plasmid or transposon or wherein the sequences are incorporated into the genome of the cell).

Any methods encompassed herein may further comprise the step of engineering immune cells to express an engineered TCR comprising the α and/or β T-cell receptor (TCR) sequences, thereby producing engineered hematological neoantigen-specific immune cells. Any immune cells encompassed herein may be T cells, antigen-specific cells, activated T cells, memory cells, naïve T cells, macrophages, B cells, natural killer cells, natural killer T cells, or a mixture thereof. In some cases for the engineered TCR, the human constant region is replaced with a constant region that is not human. The engineered TCR may comprise sequences or modifications that stabilize the α and/or β chains of the engineered TCR to facilitate suitable transgenic TCR pairing. The engineered TCR may comprise a murine constant region, has swapped constant domorins of α and β chains, comprises γδ constant domains, comprises CD3zeta, comprises a single chain TCR format, or wherein the cells comprise disruption of one or more molecules of the endogenous TCR complex, such as disruption of one or more molecules of the endogenous TCR complex by use of siRNA, CRISPR, TALEN, or ZFN.

In certain embodiments, the hematological neoantigen is associated with myeloid malignancies at the malignant and/or pre-malignant phase. The neoantigen may comprise a mutation within the KRAS, NRAS, p53, BRAF, EZH2, MYD88 (NF-κB), PAX5, DNMT3A, NPM1, IDH1, IDH2, JAK2, CALR, FLT3, KMT3A, TET2, ASXL1, CEBPA, RUNX1, PTPN11, SRSF2, MLL, KIT, EZH2, SF3B1, CBL, U2AF1, BCOR, GATA2, MYC, NOTCH1, NOTCH2, CARD11, CD79A/B, CXCR4, BIRC3, TRAF3, TCF3, KLF2, PLGG1, STAT3, STAT5B, PRKCB, or ALKgene. The hematological neoantigen may be associated with lymphoid malignancies. The neoantigen may comprise a mutation within the BCR-ABL1, ETV6-RUNX1, or TCF3-PBX1 gene. The neoantigen may be from any one of Tables 1-3. In some embodiments, the α chain comprises SEQ ID NO:49 or SEQ ID NO:51 and the β chain comprises SEQ ID NO:50 or SEQ ID NO:52.

Embodiments also include hematological neoantigen-specific T cells produced by any method encompassed herein, and the cells may be comprised in a pharmaceutically acceptable excipient. Embodiments also include engineered hematological neoantigen-specific immune cells produced by any method encompassed herein. Any cells may be comprised in a pharmaceutically acceptable excipient. In some embodiments, the TCR is modified to increase TCR affinity and/or avidity and/or functional avidity to target the neoantigen, such as by the TCR being modified by substitution of one or more amino acids in the complementarity-determining region. In specific cases, the cells comprise more than one engineered TCR. In a certain aspect, two or more engineered TCRs target different neoantigens, tumor antigens, or viral antigens. In specific cases, the cells comprise one or more heterologous molecules other than the engineered TCR and/or wherein the cells comprise disruption of one or more endogenous genes to the cells. The heterologous molecule may be a transgenic receptor; a modulator that enhances persistence and/or function of the cells; provides anticancer activity, or a combination thereof. A transgenic receptor may be a chimeric antigen receptor, a TCR targeting a non-neoantigen tumor-associated antigen, a cytokine receptor, or a chimeric cytokine receptor. A modulator may be a cytokine or cytokine receptor, chemokine receptor, or chemokine. The heterologous molecule may provide an additive or synergistic antitumor effect compared to the antitumor effect of the cells in the absence of the heterologous molecule. The cells may be further defined as being specific for another antigen than the neoantigen. In a specific embodiment, the antigen other than the neoantigen is a germline tumor antigen, a viral antigen, or wherein the cells are alloreactive T cells. Any cells may be comprised in one or more cryoprotectants. The cells may or may not be housed in a depository. Any cells may comprise a safety switch (CD20 or truncated EGFR) or suicide gene (e.g., inducible iCas9).

In some embodiments, there is a method of treating an individual with hematological cancer or a hematological precancerous condition, comprising the step of administering to the individual a therapeutically effective amount of any hematological neoantigen-specific T cells encompassed herein, or administering to the individual a therapeutically effective amount of any one of the engineered hematological neoantigen-specific immune cells encompassed herein. The cells may be administered by injection, such as intravenously.

In some embodiments, there is a method of preventing or reducing the risk for an individual for development of hematological cancer from a precancerous condition, comprising the step of administering to the individual a therapeutically effective amount of hematological neoantigen-specific T cells encompassed herein, or administering to the individual a therapeutically effective amount of any of the engineered hematological neoantigen-specific immune cells encompassed herein. The cells may be administered by injection, such as intravenously.

Embodiments include compositions comprising the identified α and/or β T-cell receptor (TCR) polypeptides that may or may not be labeled, or wherein another molecule in the composition is labeled. Examples of labels are fluorescent, colorimetric, photochromic, radioactive, is an Fc fragment of a human or non human immunoglobulin, or is an enzyme that is activateable or deactivatiable upon contact with the neoantigen. The α and/or β T-cell receptor polypeptides may be comprised in a transgenic molecule, such as a chimeric antigen receptor, a multi-specific T cell engager, or wherein the α and/or β T-cell receptor polypeptides are conjugated to a drug (such as a hematological chemotherapeutic) and/or radiotherapeutic molecule.

Embodiments of the disclosure include methods of detecting a hematological neoantigen in a sample from an individual, comprising the step of contacting an effective amount of any composition encompassed herein with the sample, which may be a research sample or a clinical sample.

Embodiments of the disclosure include methods of diagnosing a hematological cancer or precancerous condition in an individual, comprising the step of contacting an effective amount of any composition encompassed herein with the sample, which may comprise peripheral blood and may include memory cells that are specific for the neoantigen. The individual may be suspected to have cancer. The individual may be at risk for cancer compared to the general population. In some cases, when neoantigen-specific memory cells are identified from the sample, the individual is given an effective amount of one or more cancer therapies, such as immunotherapy directed to the neoantigen. The immunotherapy may comprise engineered T cells that express the antigen binding domain or all of the TCR. The immunotherapy may comprise the antigen binding domain or all of the TCR linked to an antibody or small molecule. In specific embodiments, the contacting utilizes the antigen binding domain or all of the TCR that is labeled with a fluorescent label, colorimetric label, photochromic label, radioactive label, an Fc fragment of a human or non human immunoglobulin, or with an enzyme that is activateable or deactivatiable upon contact with the neoantigen.

In certain embodiments, there is a method of activating or costimulating immune cells, comprising the step of contacting immune effector cells with an effective amount of any composition encompassed herein. The activating may comprise cytokine signaling. The cell may be further defined as comprising a molecule that provides costimulation when in contact with a T cell, such as CD80 or CD86.

In some embodiments, there is an immunological composition, comprising a neopeptide comprising a neoantigen from KRAS, NRAS, p53, BRAF. EZH2, MYD88 (NF-□B), PAX5, DNMT3A, NPM1, IDH1, IDH2, JAK2, CALR, FLT3, KMT3A, TET2, ASXL1, CEBPA, RUNX1, PTPN11, SRSF2, MLL, KIT, EZH2, SF3B1, CBL, U2AF1, BCOR, GATA2, MYC, NOTCH1, NOTCH2, CARD11, CD79A/B, CXCR4, BIRC3, TRAF3, TCF3, KLF2, PLGG1, STAT3, STAT5B, PRKCB, or ALK or wherein the neoantigen is from a translocation from ETV6-RUNX1, CBFb/YH11, BCR-ABL1, ETV6-RUNX1, IGH-IL3, TCF3-PBX1, NUP213-ABL1, PICALM-MLLT10; those involving of MLL, or TCR locus.

In specific embodiments, there is a method of identifying an immunological composition for cancer or a precancerous condition associated with one or more neoantigens, comprising the step of assaying a plurality of peptides encompassing one or more neoantigens for immunogenicity to identify one or more peptides that elicit a T cell response from multiple donors. The method may further comprise the step of formulating the identified peptide(s) with an adjuvant and/or in a pharmaceutically acceptable excipient. The method may further comprise the step of administering a therapeutically effective amount of the formulated peptide(s) to an individual with cancer or a precancerous condition. Specific embodiments include immunological compositions produced by any method encompassed herein.

### BRIEF DESCRIPTION OF THE FIGURES

For a more complete understanding of the present invention, reference is now made to the following descriptions taken in conjunction with the accompanying figures.

FIGS. 1A-1E. Generation and characterization of neoantigen specific T cells derived from healthy donors. FIG. 1A. Reactivity of pre-stimulation PBMCs following exposure to neoantigen mastermix (peptide library encompassing 14 neoantigens) or media only (negative control), as measured by IFN-γ ELISpot. Data presented as spot forming colonies (SFC)/2×10⁵ input cells; bars and error bars indicate mean and standard error of mean (SEM), respectively. FIG. 1B. Schematic diagram of the protocol to enrich neoantigen-specific T cells. FIG. 1C. Specificity of expanded cells towards neoantigen mastermix peptides or media only as measured by IFN-γ ELISpot (n=23). FIG. 1D. Fold expansion of Neoantigen-specific T cells achieved over 3 stimulations in culture, determined by cell counting based on tryptan blue exclusion (n=17). FIG. 1E. Immunophenotyping of neoantigen-specific T cells as assessed by flow cytometry, for the expression of memory, activation and exhaustion markers (n=17).

FIGS. 2A-2B. Specificity of expanded neoantigen-specific T cells against individual stimulating neoantigens. FIG. 2A. Responses towards individual neopools (whereby each neopool covers one neoantigen) from two representative donors shown in IFN-γ ELISpot images (top) and all mNeo T cell lines in a heatmap (bottom). FIG. 2B. Proportion of neoantigen-specific T cell lines with the number of neoantigen specificities.

FIGS. 3A-3E. In depth profiling of IDH2 R140Q neoantigen-specific T cells. FIG. 3A. Deconvolution of the IDH2R140Q response to identify stimulatory 15mers shown for 2 representative donors (IFN-γ ELISpot images, left), and all other IDH2 R140Q neoantigen specific-T cell lines (heatmap, normalized to % response of the most immunogenic 15mer, right). FIG. 3B. Intracellular cytokine staining of IDH2 R140Q neoantigen-specific T cells showing presence of CD4 and CD8 responses. FIG. 3C. ELISpot showing CD4 responses towards the immunogenic 15-3 neoantigen peptide and analogous wildtype peptide for 1 donor, and HLA-blocking studies showing HLA-DR restriction (FIG. 3D). FIG. 3E. Mapping of minimal epitopes for 4 different donors, identifying 3 unique 10mers (SPN-, TIQ-, IQN-) and a 9mer (IQN-) as distinct epitopes. Analogous wildtype responses for each minimal epitope are shown below dotted line. In FIG. 3A: 15-1 is SEQ ID NO:4; 15-2 is SEQ ID NO:5; 15-3 is SEQ ID NO:6; 15-4 is SEQ ID NO: 7; and 15-5 is SEQ ID NO:8. In FIG. 3C, SEQ ID NO:6 is at the top, and SEQ ID NO:67 is at the bottom. In FIG. 3E: the peptides for the 10mers from top to bottom are SEQ ID NO:9. SEQ ID NO:10, SEQ ID NO:11, SEQ ID NO:12, SEQ ID NO:13, SEQ ID NO:14. The peptides for the 9mers from top to bottom are SEQ ID NO:15, SEQ ID NO:16, SEQ ID NO:17, SEQ ID NO: 18, SEQ ID NO:19, SEQ ID NO:20, and SEQ ID NO:21.

FIGS. 4A-4H. Functional characteristics of mutant-specific IDH2 R140Q neoantigen-specific T cells. FIG. 4A. FluoroSpot showing IFN-γ, TNF-α and Granzyme B secretion following stimulation of IDH2 R140Q neoantigen-specific T cells with the mutant specific epitope (IQN-10mer) for one representative donor (left) and summary for all donors (right). FIG. 4B. Representative FluoroSpot region from Donor 20 showing polyfunctional cells secreting two or more effector molecules. FIG. 4C. Proportion of IFN γ+secreting cells that also secrete TNF-α, granzyme B or both (left); proportion of TNF-α secreting cells that also secrete IFN-γ, granzume B. or both (right) for one representative donor. FIG. 4D. Representative FluoroSpot region showing different intensity and size of IFN-γ+spot forming colonies for single, dual and triple secretors. FIG. 4E. Standard curve correlating relative spot volume derived from the FluoroSpot assay with a traditional cytokine quantification method (Luminex). FIG. 4F. Fold increase in relative spot volume in triple and dual secretors compared to single secretors, for IFN-γ (left), granzyme B (middle) and TNF-α (right). FIG. 4G. IDH2 R140Q specific T cells lysed autologous neopeptide pulsed PHA blasts as assessed in a 6 hour ⁵¹Chromium release assay at a range of effector target ratios. FIG. 4H. Donor 20's cell line lysed peptide pulsed (IQN-10mer) autologous and allogeneic targets matched at HLA-B44, but not at other alleles in a 6 h ⁵¹Cr-release cytotoxicity assay, thus determining that this epitope is presented in context of HLA-B44. FIG. 4A includes SEQ ID NO: 1, and FIG. 4H includes SEQ ID NO:22.

FIG. 5. Schematic showing design of individual neopools and neoantigen mastermix. FLT3 D835Y Neopool includes SEQ ID NOs: 23-27, and NRAS Q61K/R Neopools includes SEQ ID NOs: 28-32 (on the left) and SEQ ID NOs: 33-37 (on the right).

FIGS. 6A-6G. Generation of Transgenic DNMT3A R882C TCR-T cells from Donor 8. Neoantigen-specific T cells from donor 8 were specific to the DNMT3A R882C mutation. FIG. 6A. The minimal epitope was mapped to 10-7 NMS peptide (left), which was mutant-specific (right). Sequences from left to right on the x-axis are SEQ ID NOs: 53-63 and 68. FIG. 6B. To select the DNMT3A R882C specific T cells for cloning, the culture was stimulated with C10-7 NMS peptide, and reactive cells were isolated using the IFN-γ capture kit. FIG. 6C. top: Using limiting dilution technique, one IFN-γ+ cell was seeded in each well of a 96 well plate, on a bed of allogenic feeders in presence of CD3 stimulators (e.g., PHA) and cytokines (e.g., IL2.) Bottom: After 2-3 weeks, the clones were examined for growth, and thriving colonies were assessed for neo-specificity using ELISpot. FIG. 6D. Specific colonies (*) were sent for TCR sequencing, which confirmed the V (D) J and CDR3 regions of the TCR alpha and beta chains for these DNMT3A R882C neo-specific T cells. SEQ ID NOs: 64-65 are provided therein, from top to bottom. (FIG. 6E.) FIG. 6F. Schematic showing the DNMT3A-R882C neo-TCR construct: Neoantigen-specific VDJ regions are linked to murine TCR constant regions; alpha and beta chains are separated by P2A sequence. FIG. 6G. T cells transduced with DNMT3A R882C-specific TCR lyse autologous peptide pulsed targets (for both DNMT3A R882C and analogous R882H peptide), while remaining inert towards wildtype pulsed or unpulsed targets. SEQ ID NO:59 and SEQ ID NO: 66 are provided therein, from top to bottom

### DETAILED DESCRIPTION

### I. Examples of Definitions

The use of the word “a” or “an” when used in conjunction with the term “comprising” may mean “one,” but it is also consistent with the meaning of “one or more.” “at least one,” and “one or more than one.” As used in the specification and claims, the singular form “a”, “an”, and “the” include plural references unless the context clearly dictates otherwise.

The compositions and methods for their use can “comprise.” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that embodiments described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

The word “or” or the phrase “and/or” means “and” or “or”. For example, “x, y, and/or z” can refer to “x” alone, “y” alone, “z” alone, “x, y, and z.” “(x and y) or z.” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the inherent variation or standard deviation of error for the measurement or quantitation method being employed to determine the value.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

“Individual, “subject,” and “patient.” are used interchangeably herein and generally refers to an individual in need of treatment. The subject can be any animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having cancer. The subject may be undergoing or having undergone cancer treatment. The “subject” or “individual,” as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants. The individual may be of any gender or race or ethnicity.

The terms “prevent”, “preventing, “prevention” and the like, as used herein, unless otherwise indicated, refers to to inhibiting the onset of hematological cancer (such as from a precancerous condition), inhibiting the onset of one or more hematological cancer symptoms, or inhibiting or one or more complications from hematological cancer. It includes inhibition of the progression to a hematological cancer from a precancerous condition of any kind. An example includes progression from a myelodysplastic syndrome to acute myeloid leukemia. It includes the administration of any of the compositions, pharmaceutical compositions, or dosage forms described herein.

The terms “treat”, “treating”, “treatment” and the like, as used herein, unless otherwise indicated, refers to reversing, alleviating, or inhibiting the process, or delaying onset of the disease, disorder or condition to which such term applies, or one or more symptoms of such disease, disorder or condition and includes the administration of any of the compositions, pharmaceutical compositions, or dosage forms described herein, or eliminating the disease, condition, or disorder. In some instances, treatment is curative or ameliorating.

The terms “administering”, “administer”, “administration” and the like, as used herein, refer to any mode of transferring, delivering, introducing, or transporting a therapeutic agent to a subject in need of treatment with such an agent. Such modes include, but are not limited to, by injection, intraocular, oral, topical, intravenous, intraperitoneal, intramuscular, intradermal, intranasal, and subcutaneous administration.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques (e.g., CRISPR) which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they express one or more heterologous genes (such as synthetic TCRs, receptors of any kind including antigen receptors, and/or cytokines) and/or the cells are engineered by having reduced expression of one or more endogenous genes, all of which case(s) the engineering is performed by the hand of man. With respect to an antigen receptor, including a TCR, the antigen receptor may be considered engineered because it comprises multiple components that are genetically recombined to be configured in a manner that is not found in nature, such as in the form of a fusion protein of components not found in nature so configured. In certain aspects, the term refers to cells that are modified with a viral or non-viral vector to transgenically express the α and β chains of a TCR that may be found in nature but are force-expressed to confer in recipient cells the ability to recognize a specific neoantigen (that is, not a native feature of that recipient cell). The combination of the α and β chains of the TCR and the recipient cell modified to express them is not found in nature.

The term “heterologous” as used herein refers to being derived from a different cell type or a different species than the recipient. In specific cases, it refers to a gene or protein that is synthetic and/or not from a natural T cell. The term also refers to synthetically derived genes or gene constructs.

The term “neoantigen” as used herein refers to a protein or peptide arising from a mutation expressed by cancer cells or precancerous cells.

The term “neoantigen-specific T-lymphocytes” or “neoantigen-specific T cell lines” or “neoantigen-specific T cells” or “neospecific T cells” are used interchangeably herein to refer to T cell lines that have specificity and potency against a hematological cancer neoantigen(s) of interest or a cancer neo-epitope. As described herein, in some cases a neoantigen or two or several neoantigens are presented to native T cells in peripheral blood mononuclear cells and the native CD4 and CD8 T cell populations expand in response to said neoantigen(s). These cells can be generated from patients with cancer or pre-cancer harboring these neoantigens or also from healthy individuals. The source of the responding T cells can be from the naïve T cell fraction or from the memory T cell pool. For example, a neoantigen-specific T cell line can recognize the neoantigen, thereby expanding the T cells specific for the neoantigen. In another example, a neoantigen-specific T cell line for a first neoantigen and a second neoantigen can recognize both the first and second neoantigens, thereby expanding the T cells specific for the first and second neoantigens. The methods of the disclosure allow for induction of CD8+ T cells, CD4+ T cells, or a combination of CD8+ T cells and CD4+ reactive T cells.

The term “neo-epitope” as used herein refers to a peptide harboring a mutated amino acid/nucleotide sequence capable of binding to class I and/or class II HLA molecules and being recognized by the T cell receptor of natural T cells or engineered cells.

“TCRs” used in this application can refer to TCR in its entirety, or just regions encompassing the antigen binding domain.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the disclosure. The term “invention” is not intended to refer to any particular embodiment or otherwise limit the scope of the disclosure. Although one or more of these embodiments may be particularly considered, the embodiments disclosed should not be interpreted, or otherwise used, as limiting the scope of the disclosure, including the claims. In addition, one skilled in the art will understand that the following description has broad applications, and the discussion of any embodiment is meant only to be exemplary of that embodiment, and not intended to intimate that the scope of the disclosure, including the claims, is limited to that embodiment.

Embodiments of the disclosure include methods and compositions that encompass use and production of neoantigen-specific TCRs that target one or more hematological neoantigens that can be utilized in individuals with hematological cancer or precancerous conditions. The disclosure also provides for steps that may be taken prior to the delivery of the cells, such as useful steps for producing T cells that are more efficacious than T cells produced by other methods.

In particular embodiments, the TCRs present in the produced T cells are utilized in one or a variety of immunotherapies or detection reagents that target the neoantigen to which the TCRs are directed. Examples of immunotherapies include at least engineered TCRs, chimeric antigen receptors, engagers, and so forth. The immunotherapies may comprise the cells that express the TCRs as part of a composition or complex.

### II. Hematological Neoantigens

The present disclosure provides cell immunotherapies that target mutations expressed only by hematological cancer cells or precancerous cells, otherwise known as neoantigens. In particular embodiments, the mutations are of any kind, including those that are the following: (1) driver mutations (i.e., mutations that cause cells to become cancer cells); (2) “hot spot” mutations (i.e., mutations that are frequently detected within one cancer type, or across many different diseases/tumor types and that occur more frequently than expected compared to a background frequency; and/or (3) a patient-specific mutations.

The nature of the mutations for the hematological neoantigen may be of any kind of mutation, including a substitution, deletion, insertion, missense, nonsense, translocation, or frameshift, as examples. In some cases, the neoantigen comprises a neo-epitope that encompasses sequence surrounding the specific mutation. For example, there may be a single substitution at one amino acid, but the neoantigen comprises a neo-epitope of one or several amino acids on the N-terminal and/or C-terminal side of the single substitution.

The neoantigen may be associated with some or all hematological cancer cells or precancerous cells in an individual. Samples from an individual identified as having a hematological cancer or precancerous condition may be assayed for the presence of any type of neoantigen prior to determining whether or not the individual should be administered therapies encompassed herein.

The hematological neoantigen may be associated with one or more particular genes, and those one or more genes may or may not be known to be prone to having hematological cancer-causing mutations. In certain embodiments, more than one hematological neoantigen is known to be present in one or more genes for a hematological cancer, and the individual may or may not be subject to methods of determining whether or not the more than one neotigen is present in the cells of the individual.

In specific embodiments, the hematological neoantigen is associated with a particular gene. Examples of genes in which the neoantigen may reside include at least KRAS, NRAS, p53, BRAF. EZH2, MYD88 (NF-κB), PAX5, DNMT3A, NPM1, IDH1, IDH2, JAK2, CALR, FLT3, KMT3A, TET2, ASXL1, CEBPA, RUNX1, PTPN11, SRSF2, MLL, KIT, EZH2, SF3B1, CBL, U2AF1, BCOR, GATA2, MYC, NOTCH1, NOTCH2, CARD11, CD79A/B, CXCR4, BIRC3, TRAF3, TCF3, KLF2, PLGG1, STAT3, STAT5B, PRKCB, and ALK. Examples in which neoantigens can arise due to translocations include: ETV6-RUNX1, CBFb/YH11, BCR-ABL1, ETV6-RUNX1, IGH-IL3, TCF3-PBX1, NUP213-ABL1, PICALM-MLLT10; those involving of MLL, and TCR locus.

Examples of specific hematological neoantigens to which the TCRs are directed are provided in Tables 1, 2, or 3 herein.

TABLE 1
Lymphoma “hot spot” mutations

| Protein | Lymphoma | |
|---|---|---|
| Mutated | Type | Sequence |
| KRAS | CLL, Mantle | VVVGAVGVG(G) (SEQ ID NO: 38) |
| | Cell | VVVGADGVG(G) (SEQ ID NO: 39) |
| NRAS | CLL | ILDTAGREEY(Q) (SEQ ID NO: 40) |
| p53 | T cell, | VVPCEPPEV(Y) (SEQ ID NO: 41) |
| | DLBCL, CLL, | |
| | Mantle Cell, | |
| | Burkitt, HL | |
| Bcr-abl | ALL | SSKALQRPV (translocation) (SEQ ID |
| | | NO: 42) |
| | | GFKQSSKAL (SEQ ID NO: 43) |
| | | ATGKQSSKALQRPVAS (SEQ ID NO: 44) |
| BRAF | Hairy Cell | EDLTVKIGDFGLATEKSRWSGSHQFEQLS |
| | | (V) (SEQ ID NO: 44) |
| EZH2 | Follicular, | NDFVVDGTRKGNKI(A) (SEQ ID NO: 45) |
| | DLBCL | |
| MYD88 | DLBCL, LPL | RPIPIKYKAM(L) (SEQ ID NO: 46) |
| (NF-KB) | | HQKRPIPIKY(L) (SEQ ID NO: 47) |
| PAX5 | HL, B-ALL | SSYSISGILSITSPSA(G) (SEQ ID NO: 48) |

The site of mutation in this table is underlined, and the bracketed letter indicates the normal amino acid that is substituted by the underlined amino acid as a result of the mutation shown in the sequence

### III. Hematological Neoantigen Peptide Library

Methods of the disclosure utilize hematological neopeptide libraries as a means to produce hematological neoantigen-specific T cells having T-cell receptors (TCRs) that recognize at least one hematological neopeptide. Hematological neoantigen peptide libraries are a collection of peptides that represent a particular hematological neoantigen or, in some cases, a library may be considered to comprise a collection of peptides that represent more than one particular hematological neoantigens. The neoantigen comprises at least one mutation, and the mutation may or may not be located at different positions within the collection of peptides. A given mutation may be successively staggered among a collection of peptides. Among a collection of peptides, a given mutation may be present on a first peptide at a first position, present on a second peptide at a second position that is adjacent to the first position, present on a third peptide at a third position that is adjacent to the second position, present on a fourth peptide at a fourth position that is adjacent to the third position, and so on. The mutation may be at or towards one end of the peptide, or the mutation may be generally centrally located within the peptide. In a specific case, a single peptide (e.g., 8-30 amino acids in length) is generated and the mutation is generally centrally located within the peptide. The library comprises a plurality of peptides at least some of which are non-identical, in specific cases, and such a library may be referred to as a pepmix. In some cases, a pepmix comprises two or more libraries, such as when each of the libraries comprise a collection of pepmixes that are directed to different neoantigens, and at least some of the peptides are non-identical within the libraries.

The peptides may be said to represent a particular hematological neoantigen by each of the peptides comprising sequence of at least some of the hematological neoantigen. Collectively, the pepmix may comprise the entire region of the hematological neoantigen such that each peptide within a pepmix comprises the mutated region at a different location, in some cases. In particular embodiments, each of the peptides comprise a specific mutation sequence of the neoantigen. Some of the peptides may comprise the mutation sequence at or towards an N-terminal end of the peptide, at or towards a C-terminal end of the peptide, between the center and the end of the peptide, or approximately in the center of the peptide. In specific cases, each mutated neoantigen sequence of a given library may be located at different positions among the individual peptides, although some of the peptides in a given library may be identical or substantially identical (e.g., greater than about 75, 80, 85, 90, or 95%) to one another.

Pepmixes utilized in the disclosure may or may not be from commercially available peptide libraries. The peptides may be 15 amino acids long, or about 15 amino acids long, and they may overlap one another, such as by 11 amino acids, or by about 11 amino acids, in certain aspects. In some cases, they may be generated synthetically. Examples include those from JPT Technologies (Springfield, VA) or Miltenyi Biotec (Auburn, CA). In particular embodiments, the neoantigen peptides are at least (about) 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least (about) 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In some embodiments, the amino acids as used in the pepmixes have at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In some embodiments, the amino acids as used here in the pepmixes have at least 70% purity.

The mixture of different peptides may include any ratio of the different peptides, although in some embodiments each particular peptide is present at substantially the same numbers in the mixture as another particular peptide. The methods of preparing and producing pepmixes for multiviral cytotoxic T cells with broad specificity is described in US2018/0187152, which is incorporated by reference in its entirety.

### IV. Generation of Hematological Neoantigen-Specific T Cells

The disclosure encompasses methods of producing hematological neoantigen-specific T cell therapy using methods that are enhanced compared to known methods. The methods generate neoantigen-specific T cells that target at least one neoantigen, including 1, 2, 3, 4, 5, or more neoantigens.

The methods allow for utilizing one or more libraries of neopeptides (peptide comprising part or all of a neoantigen) that span a given neoantigen or libraries of neopeptides each of which span a given neoantigen. The methods generally utilize steps of producing neoantigen-specific T cells, including with high specificity and cytotoxicity for the neoantigen without wild type sequence reactivity using optimized culturing methods, followed by (1) administering the produced neoantigen-specific T cells alone or in combination with other immune based therapies including cancer testis/tumor-associated antigen-specific T cells, virus-specific T cells, BiTEs, etc., to an individual in need thereof; or (2) generating engineered T cells (or any other effector immune cell including NK cells, NK-T cells, macrophages, etc.) having TCRs based on the sequence of the α and β chains of the TCR of the effective neoantigen-specific T cells; (3) utilizing the sequence of the α and β chains of the TCR of the effective neoantigen-specific T cells for diagnosing tumor or pre-malignant cells expressing that mutation; or (4) manufacturing a molecule (like a chimeric antigen receptor or a bispecific T cell engager or coupled with an imaging-related molecule or chemotherapeutic, etc.) that contains the neo-antigen binding TCR for detection or immunotherapy of tumors.

The neoantigen-specific T cells may be produced by contacting a population of antigen presenting cells (APCs) (e.g., dendritic cells (DCs), B cells, monocytes, or a mixture thereof) with an overlapping library of peptides or libraries of peptides (either or which may be referred to as a pepmix). The peptides of a given library encompass a hematological neoantigen (a mutated neoantigen sequence), and in specific embodiments the mutated sequence is located at different positions within each of the individual peptides. This allows for enhanced epitope processing and presentation of the immunogenic epitope peptide as compared to when all of the peptides in the library are substantially identical. In some cases, the APCs are exposed to multiple libraries at substantially the same time, and each of the libraries encompass peptides that span a different neoantigen from another neoantigen. Such contact between the APCs and the pepmix produces pepmix-loaded APCs.

Following this, the pepmix-loaded APCs are exposed to a sufficient amount of peripheral blood mononuclear cells (PBMC) from any individual (including an individual in need of the cell therapy of the method or another individual or individuals that are not in need of the therapy). Such exposure between the two cell populations occurs under suitable conditions in vitro to produce the desired neoantigen-specific T cells, and this step utilizes conditions that stimulate the production of the neoantigen-specific T cells that are capable of responding to at least one neopeptide.

In alternative embodiments, instead of exposing the pepmix to APCs to produce pepmix-loaded APCs and then exposing the pepmix-loaded APCs to PBMCs, one can exclude the APC exposure and instead expose the pepmix library or libraries to the PBMCs directly, given the presence of APCs in the PBMCs (e.g., B cells, monocytes, etc.). In both cases, one or more stimulation steps are utilized to produce neoantigen-specific T cells.

PBMCs may be isolated from an individual in need of treatment or diagnosis or from a healthy donor, including a donor that does not have cancer or does not have a precancerous condition. The isolation of the PBMCs may occur by any suitable method, and, by way of example, it may be density centrifugation (gradient) (Ficoll-Paque), using cell preparation tubes (CPTs), or using SepMate tubes with freshly collected blood. The PBMC can comprise lymphocytes, monocytes, and dendritic cells. The lymphocytes can include T cells, B cells, and NK cells. In some embodiments, the PBMCs as used herein may be cultured or cryopreserved. In some embodiments, the process of culturing or cryopreserving the cells can include contacting the cells in culture with one or more neoantigens under suitable culture conditions to stimulate and expand neoantigen-specific T cells.

In some embodiments, the process of culturing or cryopreserving the cells can include contacting the cells in culture with one or more neo-epitopes related to one or more neoantigens under suitable culture conditions. In some embodiments, contacting the PBMCs with one or more neoantigens, or one or more neo-epitopes from one or more neoantigens, stimulates and expands a population of neoantigen-specific T cells from the respective donor's MNCs or PMBCs.

Any stimulation step employs particular medium and particular tissue culture vessels or substrates. The vessels may be flasks or tissue culture-treated plates or non tissue culture-treated plates, and the vessels may or may not comprise a gas permeable membrane. In some cases, the stimulation occurs in the presence of one or more particular media components, including one or more particular cytokines. In some cases, a specific combination of cytokines is utilized, such as IL-7, IL-12, IL-15 and IL-6. There may or may not be a second or more stimulation step, and in such cases the media and/or vessel may or may not be the same as was utilized in the initial stimulation. A second or subsequent stimulation step may utilize a different combination of cytokines compared to a first stimulation step, and one or more cytokines in different stimulations may or may not overlap. In specific cases, a second or subsequent stimulation step utilizes media that comprises IL-7 and one or both of IL-15 and IL-2.

The neoantigen-specific T cell lines may have one or more certain characteristics, including comprising CD4+ T cells and/or CD8+ T cells. In a specific case, the neoantigen-specific T cell lines are predominantly CD8+ and comprise T cells derived from both central (CD62L+) and effector memory (CD62L−) populations. The neoantigen-specific T cells may produce one or more Th1 cytokines, including when stimulated by the neoantigen(s). Examples of the cytokines include IFN-gamma, IL-2, IL-10, and TNF-alpha/beta. In some embodiments, the neoantigen-specific T cells do not significantly lyse non-cancer cells in the recipient individual. The neoantigen-specific T cell lines may or may not be cryopreserved following production.

Once the neoantigen-specific T cells have been stimulated for expansion, they may be characterized or assessed, such as for neoantigen specificity and/or cytotoxicity. In specific embodiments, the neoantigen-specific T cells are assessed for secretion of appropriate effector molecules when stimulated with the appropriate neopeptide(s) but not the corresponding wild-type sequence peptide (or the wild-type peptides stimulates to a lesser degree). In specific cases, the effector cytokines include IL-2, TNF-α, IFNγ, and/or Granzyme B. Assays that may be employed include ELIspot, Flurospot and intracellular cytokine assays, and specifically killed peptide-pulsed autologous or HLA-matched or partially HLA-matched neoantigen-expressing target cells in traditional Cr51 release assays. In specific embodiments, the HLA-restriction of the neoantigen-specific T cells is determined.

### Engineering of Cells

In some embodiments, the neoantigen-specific T cells produced by methods encompassed herein are utilized themselves as compositions for the diagnosis, prevention and/or treatment for an individual in need thereof. In other cases, the neoantigen-specific T cells produced by methods encompassed herein are not themselves utilized as a therapeutic composition, but sequences of the TCRs of these produced cells are identified and further utilized in detection, therapeutic, or preventative compositions, such as other immunotherapeutic compositions including cells. In some embodiments, the TCRs of the neoantigen-specific T cells are analyzed for their sequence, and the sequence of the TCRs is utilized in subsequently engineered immune cells or other immunotherapeutic compositions having these TCR (or similar, such as greater than 75, 80, 85, 90, or 95% identity) sequences. In specific cases, neoantigen-specific T cells known or demonstrated to be responsive to neopeptides, and not their corresponding wild-type peptides, will have their respective TCR sequences obtained. T cells then are engineered to express the neoantigen-specific TCR to produced engineered T cells (that may also be referred to as engineered neoantigen-specific T cells). The identified α and β TCR sequences may be cloned into an appropriate vector (viral or non-viral) and transduced or transfected into T or other immune (e.g., NK/NKT) cells. The multi-cistronic vectors for TCR cloning may have different orientations (e.g. TCR α sequences followed by β, or vice versa), and/or utilize IRES elements or 2A. Surface expression may be confirmed, such as by flow cytometry. Specificity may be confirmed, such as by cytokine (IL-2, TNF-α, IFNγ, and/or Granzyme) release upon exposure to the neopeptide but not the corresponding wild-type sequence. Cytotoxicity may be confirmed, such as by killing of neopeptide-pulsed targets in a Cr51 release assay. HLA-restriction can be confirmed by killing of antigen and HLA matched targets.

For the engineered T cells expressing the neoantigen-specific TCR, measures may be taken to avoid mispairing with native TCR sequences in the T cells being engineered or to enhance antitumor effectiveness (by adjusting the affinity or avidity of the TCR). For example, the constant regions for the engineered TCRs may be non-human, including murine. In addition, or alternatively, one may include sequences in the TCR that stabilize neo-TCR α and β chains to guarantee appropriate transgenic TCR pairing. Examples include the addition of disulphide bonds, hydrophobic modifications, swapping constant domains of a and β chain, disrupting endogenous TCR expression (e.g. via Gene knockouts, ZFNs, TALENs, siRNA, CRISPR, etc.), use of γδ constant domains, incorporation of CD3zeta to TCR, linking TCR to CD28 and/or CD3z, codon optimization of transgenic TCR gene, utilizing a leucine zipper-like domain, orthotopic TCR replacement using CRISPR-Cas9, use of single chain TCR format, removal of N-glycosylation sites, specific insertion of the TCR into the TCR locus of the target cells or the CD3 zeta chain or any other gene within the TCR signaling complex, expression of transgenic TCRs in gamma-delta T cells/hematopoietic stem cells, use of TCR deleted cells, and others. Adjustment of affinity and avidity can be made by specific point mutations within the TCR sequence.

For the present disclosure, examples of specific TCR alpha and beta chains produced by methods of the disclosure are provided below:

IDH2 R140Q neo-TCR

### Alpha Chain (VJ Regions):

(SEQ ID NO: 49)
MVKIRQFLLAILWLQLSCVSAAKNEVEQSPQNLTAQEGEFITINCSYSV
GISALHWLQQHPGGGIVSLFMLSSGKKKHGRLIATINIQEKHSSLHITA
SHPRDSAVYICGGSGAQKLVFGQGTRLTINP

### Beta Chain (VDJ Regions):

(SEQ ID NO: 50)
MGPQLLGYVVLCLLGAGPLEAQVTQNPRYLITVTGKKLTVTCSQNMNHE
YMSWYRQDPGLGLRQIYYSMNVEVTDKGDVPEGYKVSRKEKRNFPLILE
SPSPNQTSLYFCASSLGRGNEQYFGPGTRLTVT

### DNMT3A R882C and R882H-Specific Neo-TCR

### Alpha Chain (VJ Regions):

(SEQ ID NO: 51)
MKSLRVLLVILWLQLSWVWSQQKEVEQNSGPLSVPEGAIASLNCTYSDR
GSQSFFWYRQYSGKSPELIMFIYSNGDKEDGRFTAQLNKASQYVSLLIR
DSQPSDSATYLCAVINDYKLSFGAGTTVTVRA

### Beta Chain (VDJ Regions):

(SEQ ID NO: 52)
MLSPDLPDSAWNTRLLCRVMLCLLGAGSVAAGVIQSPRHLIKEKRETAT
LKCYPIPRHDTVYWYQQGPGQDPQFLISFYEKMQSDKGSIPDRFSAQQF
SDYHSELNMSSLELGDSALYFCASSLRQGKETQYFGPGTRLLVL

In any cells encompassed herein, including the neoantigen-specific T cells produced by methods encompassed herein and that are used for therapy, or cells that are engineered to express the TCRs of the neoantigen-specific cells produced by methods encompassed herein, may be modified by the hand of man to have one or more modifications. The modifications may be useful to confer or increase the therapeutic (and/or preventative) efficacy of the cells against neoantigens, broaden efficacy to target other tumor or viral antigens, to increase the proliferation, increase the persistence of the cells, and/or safety of the cells. The cells may be modified to express one or more molecules that are not endogenous to the cells, such as one or more synthetic receptors, such as chimeric antigen receptors, TCRs, chimeric cytokine receptors, cytokine receptors, chemokine receptors, dominant negative receptors, inverted cytokine receptors, safety switches, and so forth. In some cases, the cells are modified to express one or more recombinant proteins (e.g., cytokines, chemokines, inducible suicide constructs, such as CD20/truncated EGFR, iCas9). Such modifications may be modifications such that the non-endogenous molecule(s) is integrated into the cell genome (and this integration may or may not be directed to a specific locus), or the non-endogenous molecule(s) may be present on a vector (viral or non-viral) in the cell.

In particular embodiments, any cells are modified to express one or more antigen-specific receptors, including chimeric antigen receptors and/or antigen-specific TCRs (that are not the TCRs specific for the neoantigen). In such cases, the antigen may be a cancer antigen or a viral antigen or an antigen for another pathogen. The antigen-specific receptors may be tailored specifically to target an antigen associated with a cancer of an individual or tailored to target an antigen associated with a virus of an individual. In specific embodiments, the antigen associated with the cancer is PRAME, WT1, SSX2, MAGE, Survivin, Cyclin-A1, BCL-2, CML28, CML66, BRAP. Cyline B1, Cylin E, CYP1B1, hTERT, HOXA9, mesothelin, myeloperoxidase, MUC1, Proteinase 3, RGS5, RHAMM, MAGE, PASD1, RAGE-1, Lwis Y antigen, a-galactosylceramide, MOTCH, CD44v6, phosphopeptides, protein tyrosine phosphatase, CLL-1, CD19, CD20, BCMA, CD96, CD33, CD123, CD34, IL12RB1, B7H3, CD70, or CD99. In specific embodiments, the antigen associated with a virus is EBV, HHV6, CMV, Adenovirus, BK virus, RSV, Influenza, parainfluenza, rhinovirus, hMPV, PIV, SARS-COV-2, coronavirus, Hepatitis B, Hepatitis C, HPV, or varicella zoster virus.

TCRs can be expressed into non-engineered cells with tumor antigen or virus specificities—e.g. PRAME, WT1, SSX2, MAGE, Survivin, Cyclin-A1, BCL-2, CML28, CML66, BRAP, Cyline B1, Cylin E, CYP1B1, hTERT, HOXA9, mesothelin, myeloperoxidase, MUC1, Proteinase 3, RGS5, RHAMM, MAGE, PASD1, RAGE-1, Lewis Y antigen, a-galactosylceramide, MOTCH, CD44v6, phosphopeptides, protein tyrosine phosphatase, CLL-1, CD19, CD20, BCMA, CD96, CD33, CD123, CD34, IL12RB1, B7H3, CD70, or CD99. In specific embodiments, the antigen associated with a virus is EBV, HHV6, CMV, Adenovirus, BK virus, RSV, Influenza, parainfluenza, rhinovirus, hMPV, PIV, SARS-COV-2, coronavirus, Hepatitis B, Hepatitis C, HPV, or varicella zoster virus,

In some embodiments as an alternative to, or in addition to, the cells expressing one or more non-endogenous molecules, the cells may have disruption of one or more endogenous genes. The disrupted gene(s) may be of any kind and the disruption may be produced by any method, including CRISPR, for example. In some cases, the gene that is disrupted is the endogenous TCR of the T cell, PD-1, TIM-3, or other exhaustion markers.

### V. Therapeutic or Diagnostic Compositions and Uses Thereof

Embodiments of the disclosure include therapeutic compositions that are of the following: (1) hematological neoantigen-specific T cells produced by methods encompassed herein; or (2) immunotherapy-directed compositions that utilize TCR sequences identified from hematological neoantigen-specific T cells produced by methods encompassed herein.

In cases wherein the immunotherapy-directed compositions utilized TCR sequences from the neoantigen-specific T cells, the α chain and/or β chain sequences from the TCR may be utilized. In specific cases, the α chain and/or β chain sequences from the identified TCR are utilized in other cells of any kind that are engineered to express the α chain and/or β chain polypeptides. In some embodiments, immune cells of any kind are engineered to express part (the antigen binding portion) or all of the α chain and/or β chain polypeptides from the TCR from the hematological neoantigen-specific T cells produced by methods encompassed herein. As one example, following production of hematological neoantigen-specific T cells produced by methods encompassed herein, the α chain and/or β chain sequences from the TCR of these cells are sequenced and cloned into an appropriate viral (adenoviral, adeno-associated viral, retroviral, or lentiviral) or non-viral vector (plasmid, transposon, etc.). Vector(s) encoding the α chain and/or β chain are transfected or transduced into a desired cell, such as an immune cell, and including a T cell, natural killer (NK) cell, NK T cell, dendritic cell, antigen presenting cell, macrophage, tumor infiltrating lymphocyte, B cell, and so forth. These cells expressing the α chain and/or β chain may then be utilized as immunotherapy-directed compositions themselves, although they may be expanded prior to therapy. The cells expressing the α chain and/or β chain may or may not be stored prior to use, including frozen in a cryoprotectant. In some cases, the cells expressing the α chain and/or β chain are delivered in a therapeutic amount to an individual in need thereof, including an individual that has a hematological cancer, has a precancerous condition, or is at risk for a hematological cancer. When the cells are administered to an individual in need thereof, they may or may not be administered in conjunction with another therapy, such as chemotherapy, radiation, surgery, hormone therapy, small molecules, and the like. These cells can be given in an autologous manner or in an allogeneic manner. The produced cells may be suitably stored as a third party/off-the-shelf for use in a recipient individual that is allogeneic to the source of the cells.

In some embodiments, when the immunotherapy-directed composition comprises cells engineered to express the α chain and/or β chain, the cells that express the α chain and/or β chain may be modified in any other manner. In some cases, the cells that express the α chain and/or β chain may be modified to express one or more other heterologous sequences, including one or more receptors, including antigen receptors. The one or more other heterologous sequences may include a chimeric antigen receptor; a T cell receptor other than an endogenous TCR and other than a receptor that may utilize the α chain and/or β chain identified from the neoantigen-specific T cells; a cytokine receptor, a chimeric cytokine receptor, a cytokine, and so forth. In addition to this, or as an alternative to this, the cells that express the α chain and/or β chain may have disruption of one or more endogenous genes. In cases wherein the cells that express the α chain and/or β chain may have disruption of one or more endogenous genes, the disruption may be targeted to knock out a particular gene at a locus. In some cases, the disruption integrates a heterologous sequence into a desired locus, such as an engineered receptor sequence that knocks out a particular gene to improve activity and/or persistence of the cell.

In some embodiments, the α chain and/or β chain identified from the produced neoantigen-specific T cells are utilized in other therapeutic compositions than as being expressed by cells. In some embodiments, the α chain and/or β chain are linked or conjugated either covalently or molecular connections like disulfide bonds to one or more drugs, such as in a drug conjugate with the chain(s), or radiotherapeutic molecule. In such cases, the α chain and/or β chain targets to the drug to cancerous or precancerous cells that express the neoantigen to which the α chain and/or B chains binds, resulting in killing of the cell. The drug(s) to which the α chain and/or β chain are linked may of any kind, but in specific embodiments the drug is one or more hematological cancer drugs. Examples include drugs for Acute Lymphoblastic Leukemia (ALL): Arranon (Nelarabine); Asparaginase Erwinia Chrysanthemi; Asparaginase Erwinia Chrysanthemi (Recombinant)-rywn; Asparlas (Calaspargase Pegol-mknl); Besponsa (Inotuzumab Ozogamicin); Blinatumomab; Blincyto (Blinatumomab); Calaspargase Pegol-mknl; Cerubidine (Daunorubicin Hydrochloride); Clofarabine; Clolar (Clofarabine); Cyclophosphamide; Cytarabine; Dasatinib; Daunorubicin Hydrochloride; Dexamethasone; Doxorubicin Hydrochloride; Erwinaze (Asparaginase Erwinia Chrysanthemi); Gleevec (Imatinib Mesylate); Hyper-CVAD; Iclusig (Ponatinib Hydrochloride); Inotuzumab Ozogamicin; Imatinib Mesylate; Kymriah (Tisagenlecleucel); Marqibo (Vincristine Sulfate Liposome); Mercaptopurine; Methotrexate Sodium; Nelarabine; Oncaspar (Pegaspargase); Pegaspargase; Ponatinib Hydrochloride; Prednisone; Purinethol (Mercaptopurine); Purixan (Mercaptopurine); Rubidomycin (Daunorubicin Hydrochloride); Rylaze (Asparaginase Erwinia Chrysanthemi [Recombinant]-rywn); Sprycel (Dasatinib); Tisagenlecleucel; Trexall (Methotrexate Sodium); Vincristine Sulfate; and/or Vincristine Sulfate Liposome. Examples also include drugs for Acute Myeloid Leukemia (AML): ADE; Arsenic Trioxide; Azacitidine; Cerubidine (Daunorubicin Hydrochloride); Cyclophosphamide; Cytarabine; Daunorubicin Hydrochloride; Daunorubicin Hydrochloride and Cytarabine Liposome; Daurismo (Glasdegib Maleate); Dexamethasone; Doxorubicin Hydrochloride; Enasidenib Mesylate; Gemtuzumab Ozogamicin; Gilteritinib Fumarate; Glasdegib Maleate; Idamycin PFS (Idarubicin Hydrochloride); Idarubicin Hydrochloride; Idhifa (Enasidenib Mesylate); Ivosidenib; Midostaurin; Mitoxantrone Hydrochloride; Mylotarg (Gemtuzumab Ozogamicin); Onureg (Azacitidine); Prednisone; Rubidomycin (Daunorubicin Hydrochloride); Rydapt (Midostaurin); Tabloid (Thioguanine); Thioguanine; Tibsovo (Ivosidenib); Trisenox (Arsenic Trioxide); Venclexta (Venetoclax); Venetoclax; Vincristine Sulfate; Vyxeos (Daunorubicin Hydrochloride and Cytarabine Liposome); and/or Xospata (Gilteritinib Fumarate). Examples also include drugs for Blastic Plasmacytoid Dendritic Cell Neoplasm (BPDCN): Elzonris (Tagraxofusp-crzs); and/or Tagraxofusp-erzs. Examples also include drugs Approved for Chronic Lymphocytic Leukemia (CLL): Acalabrutinib; Alemtuzumab; Arzerra (Ofatumumab); Bendamustine Hydrochloride; Bendeka (Bendamustine Hydrochloride); Calquence (Acalabrutinib); Campath (Alemtuzumab); Chlorambucil; Chlorambucil-Prednisone; Copiktra (Duvelisib); CVP; Cyclophosphamide; Dexamethasone; Duvelisib; Fludarabine Phosphate; Gazyva (Obinutuzumab); Ibrutinib; Idelalisib; Imbruvica (Ibrutinib); Leukeran (Chlorambucil); Obinutuzumab; Ofatumumab; Prednisone; Rituxan (Rituximab); Rituxan Hycela (Rituximab and Hyaluronidase Human); Rituximab; Rituximab and Hyaluronidase Human; Treanda (Bendamustine Hydrochloride); Truxima (Rituximab); Venclexta (Venetoclax); Venetoclax; and/or Zydelig (Idelalisib). Examples also include drugs for Chronic Myelogenous Leukemia (CML): Bosulif (Bosutinib); Bosutinib; Busulfan; Busulfex (Busulfan); Cyclophosphamide; Cytarabine; Dasatinib; Dexamethasone; Gleevec (Imatinib Mesylate); Hydrea (Hydroxyurca); Hydroxyurea; Iclusig (Ponatinib Hydrochloride); Imatinib Mesylate; Myleran (Busulfan); Nilotinib; Omacetaxine Mepesuccinate; Ponatinib Hydrochloride; Sprycel (Dasatinib); Synribo (Omacetaxine Mepesuccinate); and/or Tasigna (Nilotinib). Examples also include drugs for Hairy Cell Leukemia: Cladribinel; Intron A (Recombinant Interferon Alfa-2b); Lumoxiti (Moxctumomab Pasudotox-tdfk); Moxctumomab Pasudotox-tdfk; and/or Recombinant Interferon Alfa-2b. Examples also include drugs for Mast Cell Leukemia: Midostaurin; and/or Rydapt (Midostaurin). Examples also include dugs for Meningeal Leukemia: Cytarabine.

In some cases, the α chain and/or β chain identified from the produced neoantigen-specific T cells is utilized with CD3, thereby mimicking the TCR complex and allowing for activation of other T cells. The α chain and/or β chain target the complex with CD3 to cancerous cells or precancerous cells that express the neoantigen, and the complex allows for activation of T cells in the environment of the respective cancerous cells or precancerous cells.

In certain cases, the α chain and/or β chain identified from the produced neoantigen-specific T cells are utilized in diagnostic compositions or in detection compositions. In some cases, the α chain and/or β chain may be linked to a detection molecule, such as a label of some kind. In specific embodiments, the α chain and/or β chain may be linked to a molecule that is fluorescent, colorimetric, photochromic, radioactive, and so forth. In such cases, the α chain and/or β chains each or alone are linked to a label (and the α chain may have one label and the chain has another label). Such labeled TCR sequences may be used for detection for research or diagnostic purposes. As one example of a research purpose, the labeled TCR sequences may be exposed to a sample from an individual suspected of having neoantigen-specific memory cells (as a result of having cancerous cells or precancerous cells that express the neoantigen), and if the memory cells are present in the sample then the TCR sequences will bind them, indicating the presence of cancer or precancerous cells for the individual. The labeled TCR sequences may also be utilized in a histological or cytometric manner, such as for identification of the neoantigen on the surface of cells for a research purpose or for detection in a sample suspected of having cancer or precancerous cells.

### VI. Methods of Treatment or Prevention

Embodiments of the disclosure encompass methods of treatment or prevention in which a therapeutically effective amount of cells produced by methods encompassed herein are administered to an individual in need thereof. In particular embodiments, there are methods of treating or preventing cancer or its precursor lesions in an individual comprising the step of administering by any suitable route an effective amount of neoantigen-specific T cells to an individual that has cancer or has a pre-cancerous condition or is at risk for cancer. The treatment methods will treat cancer in which the cancer has one or more neoantigens to which the neoantigen-specific T cells are directed. The prevention methods will prevent cancer from pre-cancerous cells (cells that are pre-destined to become cancerous over time, such as by genetics or following exposure to certain conditions) in which the pre-cancerous cells have one or more neoantigens to which the neoantigen-specific T cells are directed or prevent cancer in individuals at high risk of developing disease (for example, progression of clonal hematopoiesis or myelodysplasia to acute myeloid leukemia). In some cases, the methods of treatment or prevention allow for killing (including by lysing) of target cells that comprise the neoantigen(s) by contacting the target cells with an effective amount of the neoantigen-specific T cells, and in specific embodiments the contacting occurs in vivo in an individual that is the recipient of the cells.

In some cases, a therapeutic composition that comprises neoantigen-specific T cells are effective against more than one neoantigen because the population of neoantigen-specific T cells in the composition are specifically generated to recognize collectively the more than one antigen. In other cases, a therapeutic composition that comprises neoantigen-specific T cells are effective against more than one neoantigen because two or more independently generated cell lines that each are directed towards different neoantigens have been mixed or combined in the therapeutic composition. In alternative cases, the two or more independently generated cell lines are used in separate formulations for the same individual and may or may not be given at substantially the same time.

In specific embodiments, the individual is known to have a hematological cancer, is suspected of having a hematological cancer, or is at a higher risk for cancer when compared to the general population (a smoker, has a personal or family history, has exposure to the sun and/or environmental carcinogens, has a cancer-associated virus, such as HPV, is obese, is older, such as older than 40, etc.). Treatment with the produced cells may ameliorate one or more symptoms of cancer, may prevent cancer, may reduce the severity of one or more symptoms, may delay onset of the cancer, or may delay or reduce the extent of metastasis of the cancer.

In certain embodiments of the present disclosure, neoantigen-specific T cells are delivered to an individual in need thereof, such as an individual that has hematological cancer. The neoantigen-specific T cells may be autologous with respect to the individual that has cancer, or the neoantigen-specific T cells may be allogeneic with respect to the individual that has cancer. In allogeneic embodiments, the neoantigen-specific T cells may be at least partially HLA matched as when utilized in an off-the-shelf approach. The cells then mediate direct anti-tumor effects and may also enhance the individual's immune system to attack the cancer cells. In an embodiment in which a transgenic TCR is utilized in an off-the-shelf approach, the HLA allele that presents the peptide that is recognized by the transgenic TCR must be present. Therefore, the engineered TCR transgenic cells can be used in an allogeneic setting for administration to individuals that express the particular HLA molecule that is presenting the neopeptide. To reduce the risk of graft rejection and enhance persistence, one could knock out the endogenous TCR (for example, using gene editing, such as CRISPR), such that any TCR present on the surface of the cell is the transgenic receptor.

In particular embodiments, the treatment methods treat cancers in which at least one neoantigen is associated. In specific embodiments, the individual has a hematological cancer harboring one or more of the neoantigens for which the cells are targeted. Types of hematological cancers include leukemia (acute lymphocytic leukemia; acute myelogenous leukemia; chronic lymphocytic leukemia; chronic myeloid leukemia, etc.), lymphoma (Hodgkin lymphoma; non-Hodgkin lymphoma; chronic lymphocytic leukaemia (CLL); small lymphocytic lymphoma; etc.) and multiple myeloma (hyperdiploid or non-hyperdiploid). In specific cases, the cancer is primary, minimal residual disease, early cancer, advanced cancer, metastatic cancer, and/or refractory or relapsed cancer, for example. The cancer may be of any stage, including stage I, II, III, or IV, for example.

The individual may utilize the treatment method of the disclosure as an initial treatment or after (and/or with) another treatment (e.g., chemotherapy, radiation, surgery, hormone therapy, and/or other types of immunotherapy). The immunotherapy methods may be tailored to the need of an individual with cancer based on the neoantigen, type, and/or stage of cancer, and in at least some cases the immunotherapy may be modified during the course of treatment for the individual. For example, if the cancer metastasizes, then the immunotherapy may change for a neoantigen associated with the metastasis or metastases. In other cases, the immunotherapy may be modified over the course of treatment for another neoantigen present in cancer cells of the individual.

In some embodiments, the present disclosure provides methods for immunotherapy comprising administering a therapeutically effective amount of the cells produced by methods of the present disclosure. In one embodiment, a medical disease or disorder is treated by transfer of cell populations produced by methods herein and that elicit an immune response. In certain embodiments of the present disclosure, cancer is treated by transfer of a cell population produced by methods of the disclosure and that elicits an immune response. Provided herein are methods for treating or delaying progression of cancer in an individual comprising administering to the individual an effective amount of neoantigen-specific cell therapy. The therapeutically effective amount of the produced cells for use in adoptive cell therapy is that amount that achieves a desired effect in a subject being treated. For instance, this can be the amount of neoantigen-specific T cells necessary to inhibit advancement or to cause regression of cancer.

In specific embodiments, immunotherapies that utilize the TCRs, including engineered transgenic TCR T cells, or other therapies linked to the TCR (e.g., small molecule drugs, antibodies, etc.) may be administered with one or more other treatments for cancer or precancerous condition, such as any chemotherapy, azacytidine, checkpoint blockades, monoclonal antibody therapies, chimeric antigen receptor-modified T cells, surgery, radiation, hormone therapy, etc.

In some embodiments, there is a method of identifying an immunological composition for cancer or a precancerous condition associated with one or more neoantigens, comprising the step of assaying a plurality of peptides encompassing one or more neoantigens for immunogenicity to identify one or more peptides that elicit a T cell response from multiple donors. In specific embodiments, in an in vitro setting, neopeptides for which there are human T cells specific for them that expand and react consistently, including for multiple donors, may be utilized in an immunological composition. The peptides may be administered to recipient individuals either naked or in combination with one or more adjuvants. The immunological composition may comprise APCs that have been made to express the sequence of the neopeptide(s) to elicit the responses in vivo (and the APCs may be generated through gene modification or by pulsing the APCs with the appropriate peptides). Any administration of immunological compositions may occur through any suitable route, including topical, intravenous, intramuscular, subcutaneous, oral, etc. The immunological composition may or may not be a vaccine. In specific embodiments, any method further comprises the step of formulating the identified peptide(s) with an adjuvant and/or in a pharmaceutically acceptable excipient. Any method may further comprise the step of administering a therapeutically effective amount of the formulated peptide(s) to an individual with cancer or a precancerous condition. Immunological compositions produced by any method encompassed herein are also contemplated.

In some cases, the individual is provided with one or more doses of the immune cells. In some embodiments, the composition as described herein is administered to the subject a plurality of times. In some embodiments, the composition as described herein is administered to the subject more than one time. In some embodiments, the composition as described herein is administered to the subject more than two times. In some embodiments, the composition as described herein is administered to the subject more than three times. In some embodiments, the composition as described herein is administered to the subject more than four times. In some embodiments, the composition as described herein is administered to the subject more than five times. In some embodiments, the composition as described herein is administered to the subject more than six times. In some embodiments, the composition as described herein is administered to the subject more than seven times. In some embodiments, the composition as described herein is administered to the subject more than eight times. In some embodiments, the composition as described herein is administered to the subject more than nine times. In some embodiments, the composition as described herein is administered to the subject more than ten times. In some embodiments, the composition as described herein is administered to the subject a number of times that are suitable for the subjects. In specific cases where the individual is provided with two or more doses of the immune cells, the duration between the administrations should be sufficient to allow time for propagation in the individual, and in specific embodiments the duration between doses is 1, 2, 3, 4, 5, 6, 7, or more days; or 1, 2, 3, 4, or more weeks; or 1, 2, 3, 4, 5, 6, 7, 8, 9, 10, 11, 12 or more months.

In specific embodiments, the cells are provided to an individual in a therapeutically effective amount (in a range from 10³ to 10¹⁰) that ameliorates at least one symptom related to neoantigen-expressing cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰ cells. Thus, in particular embodiments an individual having a particular neoantigen-positive cancer is provided once or multiple times a therapeutically effective amount of cells expressing T cells directed towards the neoantigen.

The produced cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of cells will be dependent on the subject being treated, the severity and type of the affliction, and the manner of administration. In some embodiments, doses that could be used in the treatment of human subjects range from at least 1×10³, at least 1×10⁴, at least 1×10⁵, at least 1×10⁶, at least 1×10⁷, at least 1×10⁸, at least 1×10⁹, at least 1×10¹⁰ cells/m2. In a certain embodiment, the dose used in the treatment of human subjects ranges from about 1×10⁹ to about 1×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

The present disclosure provides methods of treating or preventing cancer comprising administering to a subject in need thereof the compositions or the pharmaceutical compositions as described herein. In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight, inclusive of all ranges and subranges therebetween. In some embodiments, the T-lymphocytes are administered to the subject. In some embodiments, the subject is immunocompromised.

In a specific embodiment, a dosing schedule for TCR immunotherapies (e.g., TCR-antibody conjugated drugs/TCR-related small molecule pharmaceuticals) is as a continuous infusion for one or more days (1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more days) or one or more weeks (1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or more weeks) depending on the pK properties of the molecule.

Therapeutically effective amounts of the produced cells can be administered by a number of routes, including parenteral administration, for example, intravenous, intraperitoneal, intramuscular, intrasternal, intratumoral, intrathecal, intraventricular, through a reservoir, intraarticular injection, or infusion.

The present disclosure provides methods of lysing a target neoantigen-expressing cancer cell comprising contacting the target cell with the compositions or pharmaceutical compositions as described herein. In some embodiments, the contacting between the target neoantigen-expressing cancer cell and the compositions or pharmaceutical compositions occurs in vivo in a subject. In some embodiments, the contacting between the target cell and the compositions or pharmaceutical compositions occurs in vivo via administration of the neoantigen-specific T-cells to a subject. In some embodiments, the subject is a human.

The present disclosure provides pharmaceutical compositions comprising a polyclonal population of T-cells that recognize a plurality of neoantigens. In some embodiments, the present disclosure provides a polyclonal population of T-cells that recognize a plurality of neoantigens comprising at least one antigen from each of one or more genes.

In some embodiments, the present disclosure provides pharmaceutical compositions comprising the compositions as described herein formulated for intravenous delivery. In some embodiments, the composition as described herein is negative for bacteria. In some embodiments, the composition as described herein is negative for fungi. In some embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In some embodiments, the composition as described herein is negative for bacteria or fungi for at least 7 days in culture.

In some embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the pharmaceutical compositions formulated for intravenous delivery are negative for mycoplasma.

The cells of the disclosure may be encompassed in a pharmaceutically acceptable carrier. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### VII. Methods of Diagnosis of Pre-Cancerous or Cancer Expressing Neoantigens

Embodiments of the disclosure include methods of diagnosing a pre-cancerous condition or cancer, wherein the pre-cancerous condition or cancer is associated with one or more neoantigens (e.g., a sample from an individual suspected of having a pre-cancerous condition or cancer has cells that express the neoantigen(s)). Other methods include measuring, assaying, detecting, or analyzying in a sample for one or more particular conditions associated with a pre-cancerous cells or pre-cancerous condition or cancer. In specific embodiments, the disclosure includes methods of treatment that are determined, or the need is determined, following methods of measuring, assaying, detecting, or analyzying in a sample for one or more particular conditions associated with a pre-cancerous condition or cancer.

Endogenous T cells in individuals with neoantigen-specificity can recognize neoantigens in pre-cancerous and cancerous lesions and develop into memory cells. These neoantigen-specific memory cells circulate in peripheral blood, lymph nodes, tumor sites, bone marrow, and traffic to different organs, and their detection can indicate the presence of the cancerous or pre-cancerous lesion in the individual, including for diagnosis. Thus, in specific embodiments, there are methods of measuring for the presence of neoantigen-specific memory cells in a sample from an individual suspected of having or known to have pre-cancerous cells or a pre-cancerous condition or cancer. In some cases, there are methods of detecting neoantigen-specific memory cells in an individual suspected of having or known to have pre-cancerous cells or a re-cancerous condition or cancer. In some cases, there are methods of analyzing or assaying a sample for the presence of neoantigen-specific memory cells in a sample from an individual suspected of having or known to have pre-cancerous cells or a pre-cancerous condition or cancer. In specific embodiments, upon identification of neoantigen-specific memory cells in a sample from an individual suspected of having or known to have pre-cancerous cells or a pre-cancerous condition or cancer, the individual is appropriately treated with any suitable kind of treatment (or prevention when the individual has pre-cancerous cells or a pre-cancerous condition). The treatment/prevention may including neoantigen-specific T cells and/or one or more other cancer treatments, such as chemotherapy, immunotherapy, hormone therapy, radiation, surgery, etc.

In a specific embodiment, a sample from an individual, such as peripheral blood, is screened for the presence of known neoantigen-specific TCR sequences. In cases wherein the the TCRs are present in the memory compartment, then the individual is determined to have a pre-cancer/cancer condition or is suspected of having a pre-cancer/cancer condition and further testing may occur.

The disclosure includes methods of detecting neoantigen-specific memory cells directed against one or more neoantigens in an individual suspected of having or known to have pre-cancerous cells or a pre-cancerous condition or cancer, comprising performing a step for detecting the neoantigen-specific memory cells in a sample from the individual and, upon detection, treating the individual with a therapy for the cancer or a preventative for the pre-cancerous condition. Also disclosed herein are methods for treating cancer, or preventing onset of cancer in an individual with pre-cancerous cells or a pre-cancerous condition, the method comprising detecting neoantigen-specific T cells in a sample from the individual and administering to the individual an effective amount of neoantigen-specific T cells and/or one or more other cancer treatments, such as chemotherapy, immunotherapy, hormone therapy, radiation, surgery, etc. Methods of the disclosure include methods of treating cancer or treating a pre-cancerous condition or preventing cancer in an individual with a pre-cancerous condition, comprising the step of administering to the individual an effective amount of neoantigen-specific T cells and/or one or more other cancer treatments, such as chemotherapy, immunotherapy, hormone therapy, radiation, surgery, after determining that the individual has neoantigen-specific memory T cells in a sample from the individual.

Thus, embodiments of the disclosure encompass methods of diagnosing pre-cancer or cancer by identifying neoantigen specificity in memory cells. The memory cells can be assessed in the absence of an enrichment step in which the memory cells are first contacted with APCs that have been contacted with one or a plurality of pepmix libraries that encompass the neoantigen(s). In other cases, the memory cells can be assessed following an enrichment step in which the memory cells are stimulated with APCs that have been contacted with one or a plurality of pepmix libraries that encompass the neoantigen(s).

The memory T cells (whether or not they have undergone expansion) may be assayed for specificity. In some cases, the memory T cells may be exposed to at least one pepmix library that encompasses one or more neoantigens. Specificity of the memory T cells may be determined by secretion of one or more appropriate effector molecules when stimulated with the appropriate neopeptide(s), such as IL-2, TNF-α, IFNγ, and/or Granzyme B. Assays that may be employed include ELIspot, Flurospot and intracellular cytokine assays, and specifically killed peptide-pulsed autologous or HLA-matched neoantigen-expressing target cells in traditional Cr51 release assays. Specificities can also be determined by using neoantigen-specific multimers (HLA-peptide molecules that bind to cells expressing neoantigens with associated HL) or identification of TCR sequences with known reactivity to neopeptides.

In some embodiments of the present methods, the methods further comprise obtaining the sample from the individual. The sample may or may not be stored prior to the present methods steps.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Targeting Neoantigens Using Adoptively Transferred Neoantigen-Specific T Cells

High risk acute myeloid leukemia (AML) requires treatment with intensive chemotherapy and allogeneic hematopoictic stem cell transplant (allo-HSCT) for optimal chance of cure. However, not only do treatment-related complications such as infections and graft versus host disease contribute to significant mortality and morbidity, >50% of patients relapse, thus underscoring the need for novel, safe and efficacious therapies. In this example, an immunotherapeutic approach using T cells targeting 14 recurrent AML driver mutations in genes including DNMT3A, FLT3 and IDH2, and which are associated with poor prognosis, is characterized. It is demonstrated herein that these neoantigens are immunogenic in >70% of healthy individuals tested and in donors of diverse HLA backgrounds. Furthermore, the induced T cells, which recognized both CD4+ and CD8+ epitopes, were polyclonal and polyfunctional and exhibited cytolytic effects in vitro and in vivo. Overall, the findings show the feasibility and clinical utility of targeting neoantigens using adoptively transferred neo-T cells.

### Examples of Methods

### Healthy Donor Samples

Peripheral blood mononuclear cells (PBMCs) were isolated from healthy donors with informed consent on a Baylor College of Medicine institutional review board-approved protocol (H-36346). PBMCs were used to generate dendritic cells (DCs), neoantigen-specific T cells (neo-T cells) and phytohemagglutinin-stimulated T cells (PHA blasts), as described below.

### Neoantigen-Specific T Cells

Neoantigen peptides: The inventors examined 14 driver mutations across 8 genes in AML (Table 2). For each neoantigen, a neopool (2 mg/peptide/uL) was generated that comprised of 5 15mers overlapping by 11aa, this ensuring that the mutant amino acid was present at various positions in each peptide within the neopool. The 14 neopools spanning each of the chosen driver mutations (70 peptides total) were then combined to generate a neoantigen mastermix (FIG. 5). To generate multi-neo-T cells, T cells were stimulated with the neoantigen mastermix. For studies of individual mutations (e.g., IDH2-R140Q), T cells were stimulated using the IDH2R140Q neopool only. Minimal epitopes for CD8-mediated responses were mapped using 9mer and/or 10mer peptides overlapping by 8 or 9aa, respectively. Lypophilized peptides were purchased from Genemed Synthesis (San Antonio, TX), and reconstituted in dimethyl sulfoxide (Sigma-Aldrich, St Louis, MO).

TABLE 2
Frequency of selected mutated genes and neoantigens in
de novo acute myeloid leukemia profiled in this study.

| | % AMI | Mutation targeted for | % of total |
|---|---|---|---|
| Gene | mutated | neo-specific T cells | mutations in gene |
| FLT3 | 29% | D835Y | 28%^{a} |
| DNMT3A | 22% | R882H | 50% |
| | | R882C | 22% |
| NPM1 | 22% | W288Cfs*12 Type A/D | 70% |
| | | W288Cfs*12 Type B | 11% |
| NRAS | 12% | Q61K | 15% |
| | | Q61R | 15% |
| IDH2 | 12% | R140Q | 83% |
| IDH1 | 9% | R132H | 39% |
| JAK2 | 4.5% | V617F | 95% |
| KRAS | 4% | G12D | 25% |
| | | G13D | 17% |
| | | G12V | 13% |
| | | G12A | 8% |

Monocyte-derived DC generation: Monocytes were magnetically isolated from PBMCs using CD14 microbeads (Miltenyi Biotec, Germany), per manufacturer's instructions. These were seeded at 0.5×10⁶ cells/mL in 24 well plates in DC medium (CellGenix USA, Portsmouth, NH) supplemented with 800 U/mL granulocyte macrophage-colony stimulating factor (GM-CSF) and 400 U/mL IL-4 for 5 days. Cytokines were replenished on day 2 or 3. The immature DCs were then matured on day 5 or 6 in DC medium supplemented with 1 μg/mL prostaglandin E1 (Sigma-Aldrich), 10 ng/ml IL-1b, 10 ng/ml tumor necrosis factor α (TNF-α), 100 ng/ml IL-6, 800 U/mL GM-CSF and 400 U/mL IL-4 (all from R&D Systems, Minneapolis, MN).

In vitro expansion of neo-T cells: PBMCs were stimulated every 7-10 days using autologous neoantigen mastermix- or neopools-pulsed DCs at a ratio of up to 10 T cells/PBMCs: 1 DC. T cells were maintained in T cell medium [RMPI-1640 (Hyclone Laboratories, South Logan, UT), Click's Medium (FUJIFILM Irvine Scientific, Santa Ana, CA), 5% Human AB Serum (Valley Biomedical, Winchester, VA), and GlutaMax (Gibco, Life Technologies, Gaithersburg, MD], supplemented with IL-7 (10 ng/ml), IL-12 (10 ng/mL), IL-15 (5 ng/mL) (all from Peprotech, Rocky Hill, NJ) and IL-6 (100 ng/mL) for the first stimulation. Cultures were split and/or fresh T cell medium and cytokines replenished on day 6-8. For the second and third stimulation, T cell medium was supplemented with IL-15+IL-7 and IL-15+IL-2, respectively (R&D Systems, Minneapolis, MN), respectively, with feeding occurring on day 3 or 4.

### PHA Blasts

PHA blasts were generated from PBMCs using PHA (5 μL/mL) (Sigma-Aldrich) and maintained in T cell medium with 100 U/mL IL-2, which was replenished every 2-3 days.

### Tumor Cell Lines

The parental and IDH2 mutant TF-1 cell lines were obtained from American Type Culture Collection (ATCC) and maintained in RPMI-1640 medium supplemented with 2 ng/ml recombinant GM-CSF and 10% FBS (Hyclone Laboratories). Cell lines were authenticated by DNA short tandem repeat profiling (University of Arizona, Tucson, AZ).

### Flow Cytometry for Immunophenotypic Analysis

After 3 stimulations, neo-T cells were surface stained with the monoclonal antibodies: CD3-PerCP (BD Biosciences, Pasadena, CA), CD56-PECy7 (BioLegend, San Diego, CA), CD4-KO and CD8-PB (both Beckman Coulter, Indianapolis, IN), activation markers [CD69-ECD (BioLegend), CD28-PE and CD25-FITC (both BD Biosciences, San Jose, CA)] and exhaustion markers [PD1-PE (BD Biosciences) and TIM3-APC (BioLegend)]. Briefly, T cells were washed and pelleted, and antibodies were added in saturating amounts (2 μL). At least 50,000 live cells were acquired using Gallios flow cytometer, and data was analyzed using Kaluza software (Version 1.3, Beckman Coulter).

### Functional Assays

### ELISpot and FluoroSpot

The enzyme-linked immunospot (ELISpot) assay was used to screen and enumerate the frequency of antigen-specific IFN-γ secreting T cells, while the FluoroSpot assay (detecting IFN-γ, TNF-α, and Granzyme B) was performed to evaluate polyfunctionality. In both assays, 5×10⁵ PBMCs or 0.5-2×10⁵ neo-T cells were seeded/well in 96 well plate and exposed to individual peptides, neopools or neo-mastermix, with PHA (1 μg/mL) and unstimulated cells serving as positive and negative controls, respectively. To specifically examine CD4+ T cell responses, CD4+ cells were immunomagnetically selected using MicroBeads (Miltenyi Biotech, Bergisch Gladbach, Germany) prior to performing the ELISpot/FluoroSpot assays. For class II HLA-restriction studies, selected CD4+ cells were pre-incubated (10 μg/mL, 1 hour) with HLA-DR, -DQ (both Biolegend) or -DP (Abcam, Cambridge, MA) blocking antibodies prior to stimulation with peptides. Plates were developed as per manufacturer's instructions and analyzed using Mabtech IRIS (Stockholm, Sweden).

### Cytokine Multiplex Assay

Cytokine production was assessed using the MILLIPLEX High Sensitivity Human Cytokine Panel (Millipore, Billerica, MA), per manufacturer's instructions. Briefly, 0.5-2×10⁵ neo-T cells were stimulated with individual peptides, neopools or neoantigen mastermix. After an overnight incubation, supernatant was harvested, plated in duplicate wells, and incubated overnight at 4° C. with antibody-immobilized beads. These were then washed and plated for 1 hour at room temperature with biotinylated detection antibodies, prior to adding streptavidin-phycoerythrin for 30 minutes at room temperature. Samples were then washed and analyzed using Luminex 200 (XMAP Technology) and xPONENT Software.

### Intracellular Staining

Intracellular staining (ICS) was performed to assess for whether neoantigen-directed responses were mediated by CD4 or CD8 populations. Briefly, neo-T cells were harvested, counted and resuspended in T cell medium at 5×10⁵ cells/well in U-bottom 96 well plates. These were then stimulated with the neopool or individual peptides, or medium only in the presence of CD28 and CD49d (BD Biosciences), followed by addition of BD GolgiStop and BD GolgiPlug which contain monensin and brefeldin A, respectively. After overnight incubation, T cells were surface stained [CD8-PB, CD4-KO and CD3-PerCP, 15 minutes at 4° C.], then washed with Dulbecco's phosphate buffered saline (DPBS, Sigma-Aldrich Co.) prior to permeabilization with BD Cytofix/Cytoperm Solution (BD Biosciences). T cells were then stained with IFN-γ-APC (Biolegend) and TNF-α-PE antibodies (BD Biosciences) for 45 minutes at 4° C. At least 75,000 live cells were acquired using the Gallios flow cytometer, and analysis was performed using Kaluza software.

### ⁵¹Cr-Release Assay

The cytolytic ability of neo-T cells was assessed in a 6-hour ⁵¹Cr-release assay. Targets were tumor cell lines or autologous and partially HLA-matched, peptide-pulsed PHA blasts, with unpulsed PHA blasts as negative controls. These were labelled with ⁵¹Cr, mixed with neo-T cells at E: T ratios ranging from 80:1 to 1.25:1, and incubated for 6 hours at 37° C., 5% CO₂. All test conditions were performed in triplicate. Specific lysis was calculated by using the following formula: Specific lysis (%)=[(Experimental−spontaneous release)/(Maximum−spontaneous release)]×100. For HLA-blocking experiments, target cells were pre-incubated with HLA class I or II antibodies (10 μg/mL, Santa Cruz Biotechnology, Santa Cruz, CA) for 30 minutes prior to addition of effector T cells.

### Results

### Detection and Characterization of Neoantigen-Specific T Cells in Healthy Individuals

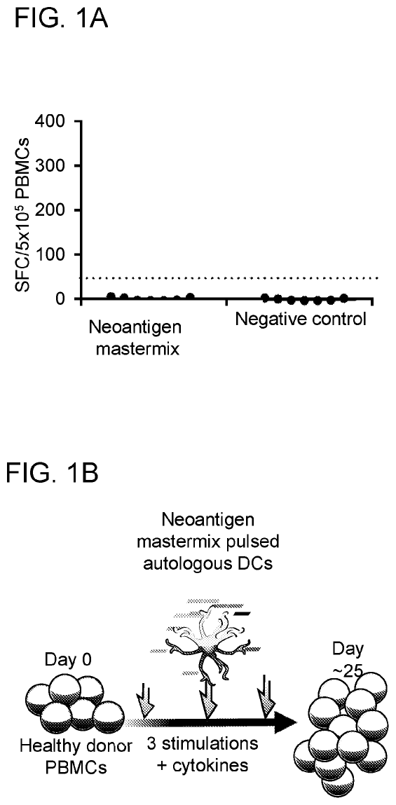

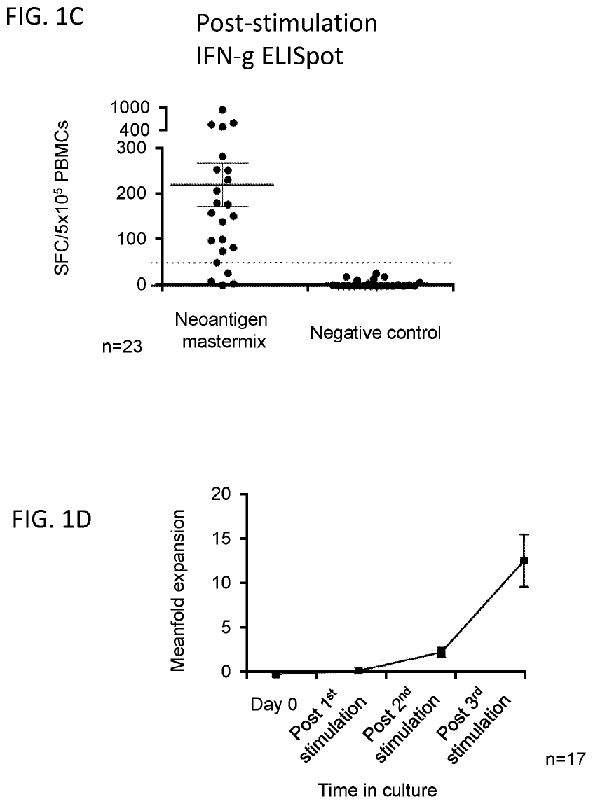

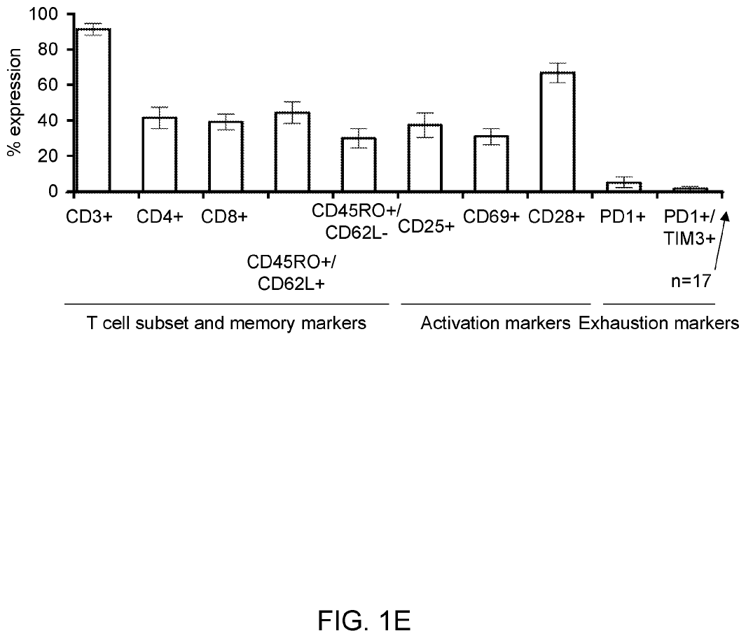

To first determine whether neoantigen-specific T cells (neo-T cells) were present at detectable levels in the circulation, the peripheral blood was examined of 23 healthy donors with diverse HLA types (Table 5) and measured immune reactivity against candidate neoantigen peptides using the IFN-γ ELIspot. As shown in FIG. 1A, neo-T cells were not detected in any of the 15 donors [4±1 spot forming colonies [SFC]/5×10⁵ PBMCs (mean±SEM); negative control 2±1 SFC/5×10⁵ PBMCs). It was considered that the inability to detect reactive PBMCs might simply reflect their low frequency and hence the inventors sought to amplify and expand any specific cells. This was achieved by performing repetitive rounds of in vitro stimulation using autologous neoantigen mastermix-loaded DCs as antigen presenting cells (APCs), and culture in the presence of pro-proliferative and pro-survival cytokines that has been previously demonstrated support the expansion of tumor-reactive T cells (Gerdemann, 2011) (FIG. 1B). After a mean of 25 days in culture neo-T cells were detected in 74% (n=17/23) of the donors (mean 279±46 SFC/5×10⁵ in responding donors; negative control 6±9 SFC/5×10⁵, FIG. 1C). In responding donors, we achieved a mean fold expansion of 13.6±3.0 (FIG. 1D). These expanded cells contained both helper (CD4+: 41.7±5.6%) and cytotoxic (CD8+: 39.4±4.6%) subsets that were activated based on expression of CD28 (37.6±6.8%), CD69 (31.2±4.5%) and CD25+ (66.9±5.5%) with minimal PD1/Tim3 expression (PD1: 5.6±2.9%, PD1+/TIM3+: 2.2±1.3%). Furthermore, both central (CD45RO+/CD62L+: 44.6±6.14%) and effector memory subsets (CD45RO+/CD62L−: 30.3±5.5%) were represented (FIG. 1E). Taken together this data demonstrates that neo-T cells are present in the circulation at low levels and can be expanded from the majority of individuals.

Neo-T Cells Target Multiple Neoantigens with Hierarchy of Immunodominance

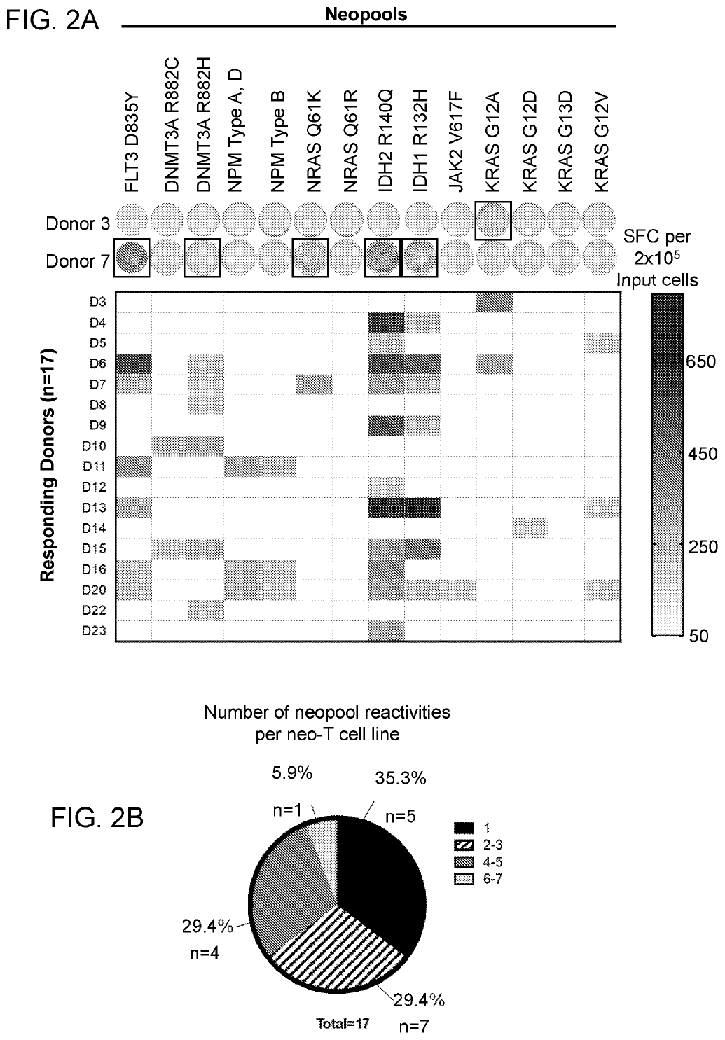

Having established the feasibility of generating neo-T cells using the neoantigen mastermix, it was next sought to identify which of the stimulating neoantigens were immunogenic. Thus, the inventors deconvoluted the mastermix and examined T cell reactivity against each of the neopools representing individual neoantigens. FIG. 2A (top) shows results from 2 representative donors-donor 3's neo-T cell response was focused solely on the KRAS G12A neoantigen (387 SFC/2×10⁵), while donor 7's neo-T cells recognized 5 different neoantigens [IDH2^{R140Q} (286 SFC/2×10⁵), NRAS^{Q61K} (229 SFC/2×10⁵), FLT3^{D835Y} (133 SFC/2×10⁵), IDH1^{R132H} (115 SFC/2×10⁵), DNMT3A^{R882H} (32 SFC/2×10⁵)]. FIG. 2A (bottom) summarizes the pattern of reactivity for each responding donor and mutation, while Table 3 ranks the immunodominance of target neoantigens (defined as the number of responding donors and frequency of reactive T cells, as measured by the IFN-γ ELISpot). Overall, all but two neoantigens (NRAS^{Q61R} and KRAS^{G13D}) were immunogenic in at least 1 donor, and the most immunodominant neoantigens were: IDH2^{R140Q} (n=11/17, 65% responding donors, 391±92 SFC/2×10⁵], IDH1 R132H (n=7/17, 41%, 295±106 SFC/2×10⁵) and FLT3 D835Y (n=6/17, 35%, 242±87 SFC/2×10⁵). Lastly, 70% of responders recognized at least 2 antigens and 50% responded to between 4 and 7 (of the 14 neoantigens in the mastermix; FIG. 2B), indicating the multi-specificity of the neo-T cells.

TABLE 3
Immunodominance of AML neoantigens, ranked by number
of responding donors and IFN-g ELISpot response.

| | # of responding | | | | |
|---|---|---|---|---|---|
| Mutation | donors (%) | | Mean ± SEM | Median (range) | |
| IDH2 R140Q | 11 | (65%) | 390.8 ± 92.3 | 286 | (770-31) |
| IDH1 R132H | 7 | (41%) | 294.6 ± 106.2 | 115 | (796-58) |
| FLT3 D835Y | 6 | (35%) | 242 ± 87.1 | 161.5 | (642-85) |
| DNMT3A R882H | 6 | (35%) | 68.5 ± 15.8 | 64 | (111-35) |
| NPM Type A,D | 3 | (18%) | 173.3 ± 28.6 | 152 | (230-138) |
| NPM Type B | 3 | (18%) | 76.3 ± 21.5 | 87 | (107-35) |
| KRAS G12V | 3 | (18%) | 42.3 ± 4.3 | 38 | (51-38) |
| KRAS G12A | 2 | (12%) | 303.5 ± 83.5 | 303.5 | (387-220) |
| DNMT3A R882C | 2 | (12%) | 36 ± 0 | 36 | (36-36) |
| NRAS Q61K | 1 | (6%) | 229 ± 0 | 229 | (229-229) |
| KRAS G12D | 1 | (6%) | 55 ± 0 | 55 | (55-55) |
| JAK2 V617F | 1 | (6%) | 45 ± 0 | 45 | (45-45) |

| | | | | |
|---|---|---|---|---|
| KRAS G13D | 0 | (0%) | — | — |
| NRAS Q61R | 0 | (0%) | — | — |

### In Depth Characterization of IDH2^{R140Q}-Specific Neo-T Cells

### Mapping of IDH2^{R140Q} Neo-Epitopes

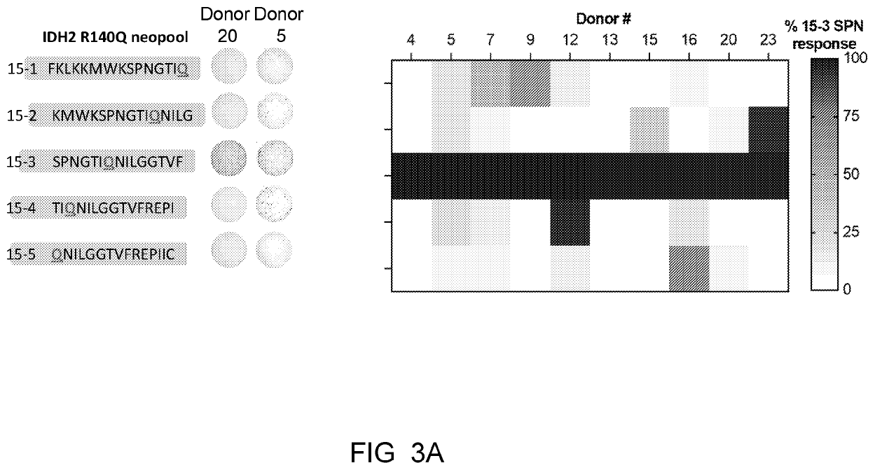

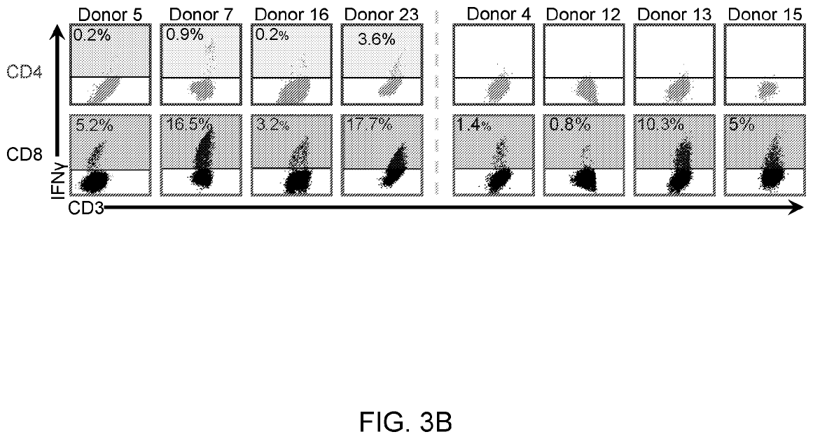

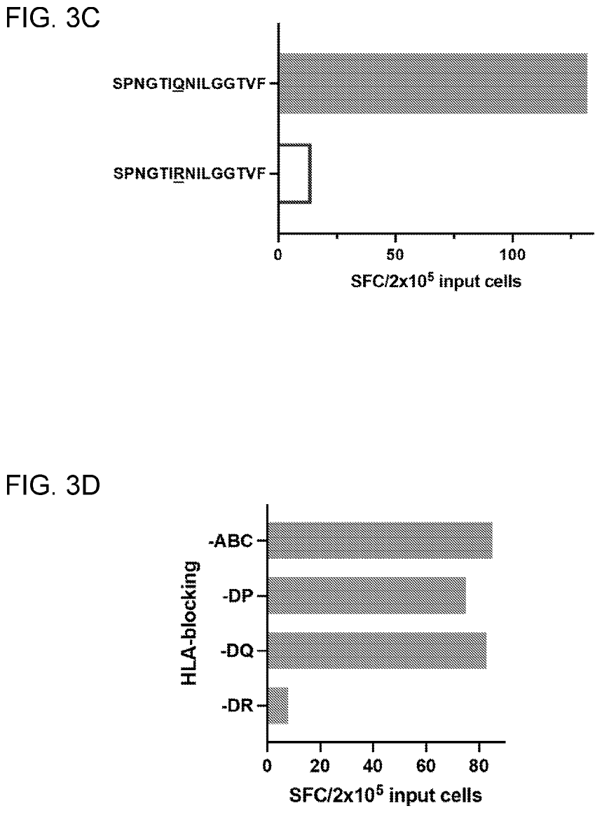

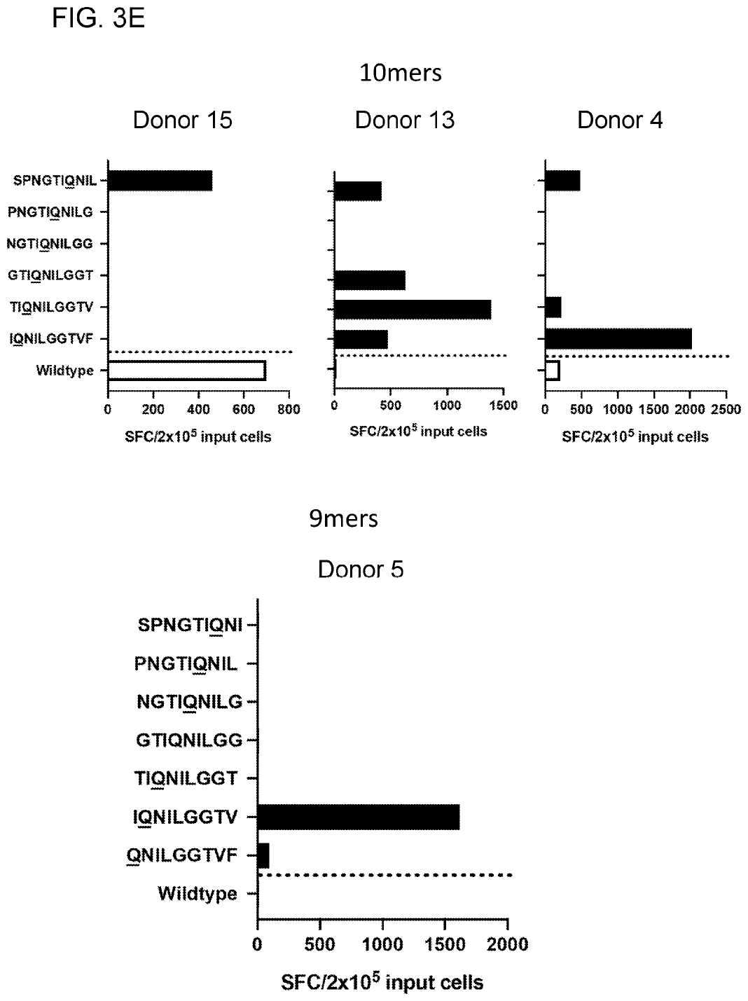

Having determined that the IDH2R140Q mutation was immunodominant, the inventors next focused on extensively profiling neoIDH2 R140Q-T cells. To first identify the immunogenic peptide within the neopool, IDH2^{R140Q} neopool-reactive T cells were exposed to each of the individual 15 mers. FIG. 3A, (center) shows results from 2 representative donors whose dominant reactivity was directed to the SPN peptide (SPNGTIQNILGGTVF, aa 134-148; SEQ ID NO:6). It was subsequently confirmed that this SPN 15mer was immunogenic in all donors who responded to this neopool (FIG. 3A, right).

To next determine whether reactive cells were derived from the CD4+ or CD8+ subsets, ICS analysis was performed following SPN peptide stimulation, and all 11 donors mounted a CD8+ response, with 4 additionally mounting a CD4+ response (FIG. 3B), suggesting the presence of overlapping HLA class I and II epitopes within this single immunogenic peptide.

To further characterize these immune responses we first focused on the CD4 responses, which were detected in donors 5, 7, 16, and 23 (HLA types shown in Table 5). The inventors sought to ascertain whether reactive T cells were mutant specific by co-culturing CD4-selected cells with both the mutant SPN 15mer and its wildtype equivalent. FIG. 3C shows the result for donor 7 s which showed a response towards the 15-3 SPN peptide (132 SFC/2×10⁵) with minimal response to the analogous wildtype peptide (14 SFC/2×10⁵). Next the inventors sought to identify the HLA class II restricting alleles for this donor (DP *04,*04; DQ *03, *04; DR *04, *08). Thus, CD4+ cells were isolated and pre-incubated with DR, DQ or DP blocking antibodies followed by stimulation with the SPN-15mer peptide. FIG. 3D shows that the reactivity was diminished in the presence of the DR blocking antibody, indicating that this Class II epitope is restricted to either DR *04, *08.

TABLE 4
Class I and Class II HLA-typing for donors
screened for neoantigen-specific T cells

| | CLASS I | | | | | | CLASS II | | | |
|---|---|---|---|---|---|---|---|---|---|---|
| Donor | HLA-A | | HLA-B | | HLA-C | | HLA-DRB1 | | HLA-DQB1 | |
| 1 | 24 | 31 | 7 | 13 | 3 | 7 | 15 | 15 | 6 | 6 |
| 2 | 2 | 30 | 13 | 38 | 6 | 7 | 7 | 16 | 2 | 5 |
| 3 | 11 | 24 | 7 | 52 | 7 | 12 | 3 | 15 | 2 | 6 |
| 4 | 11 | 68 | 7 | 35 | 7 | 12 | 3 | 7 | 2 | 2 |
| 5 | 2 | 2 | 7 | 15 | 1 | 7 | 9 | 11 | 3 | 3 |
| 6 | 2 | 30 | 13 | 35 | 6 | 8 | 7 | 8 | 2 | 6 |
| 7 | 24 | 68 | 35 | 40 | 1 | 3 | 4 | 8 | 3 | 4 |
| 8 | 2 | 11 | 15 | 40 | 1 | 2 | 8 | 9 | 3 | 4 |
| 9 | 2 | 24 | 44 | 50 | 6 | 16 | 11 | 15 | 3 | 6 |
| 10 | 3 | 3 | 7 | 27 | — | — | — | — | — | — |
| 11 | 24 | 30 | 13 | 60 | 10 | 6 | — | — | — | — |
| 12 | 2 | 24 | 35 | 39 | 4 | 7 | 9 | 11 | 3 | 3 |
| 13 | 11 | 29 | 35 | 44 | 4 | 16 | 1 | 7 | 2 | 5 |
| 14 | 11 | 24 | 8 | 52 | 2 | 12 | 3 | — | 2 | — |
| 15 | 2 | 33 | 35 | 44 | 7 | 7 | 1 | 7 | 2 | 5 |
| 16 | 2 | 2 | 15 | 15 | — | — | — | — | — | — |
| 17 | 2 | 3 | 15 | 44 | 1 | 7 | — | — | — | — |
| 18 | 24 | 33 | 7 | 58 | 3 | 7 | 1 | 3 | 2 | 5 |
| 19 | 2 | 24 | 52 | 52 | — | — | 15 | 15 | — | — |
| 20 | 11 | 68 | 8 | 44 | 7 | 7 | 3 | 11 | 2 | 1 |
| 21 | 24 | 29 | 7 | 27 | — | — | — | — | — | — |
| 22 | 2 | 3 | 35 | 13 | — | — | — | — | — | — |
| 23 | 2 | 24 | 35 | 44 | 4 | 16 | 4 | 4 | 3 | 3 |

The HLA-class I restricted response was examined. Thus, a panel was generated of 9 and 10mers (overlapping by 8 and 9aa, respectively) spanning the SPN 15mer and exposed neoIDH2 R140Q-T cell lines to each of these individual peptides, identifying a total of 4 different immunogenic minimal epitopes (FIG. 3E, above dotted line). For instance, donor 15's response mapped to the 10mer SPNGTIQNIL (SEQ ID NO:9), while neo^{IDH2 R140Q}-T cells from donors 13 and 20 recognized the 10mers TIQNILGGTV (SEQ ID NO:13) and IQNILGGTVF (SEQ ID NO: 1), respectively. Finally, the minimal epitope for donor 5 mapped to the IQNILGGTV 9mer (SEQ ID NO:1). Overall, these findings confirm the presence of multiple CD8+ neoepitopes in individuals of disparate HLA types within the IDH2^{R140Q} neoantigen.

Given that the mutant peptides differ from the wildtype counterpart by just a single amino acid, the inventors next sought to investigate whether the reactive T cells showed any activity to their wildtype counterparts. As shown in FIG. 3E (below dotted line) while immune activity to the SPN 10mer proved to be wild-type cross-reactive, the responses to the TIQ and IQN 10mers as well as the IQN 9mer proved to be highly mutant-specific, hence representing CD8-restricted peptide sequences that could be safely targeted immunotherapeutically.

### Functional Characterization of Mutant-Specific IDH2^{R140Q}-Neo T Cells

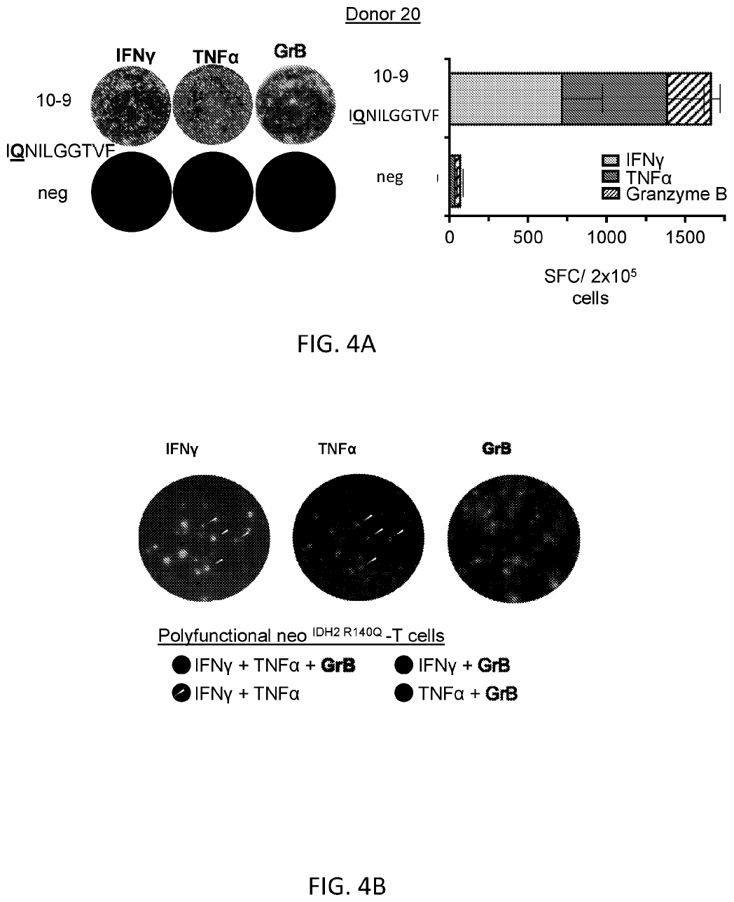

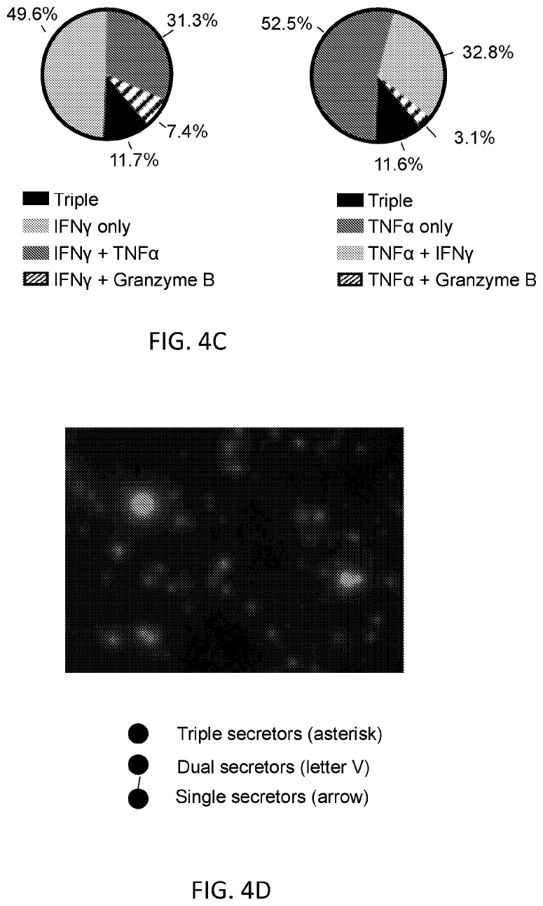

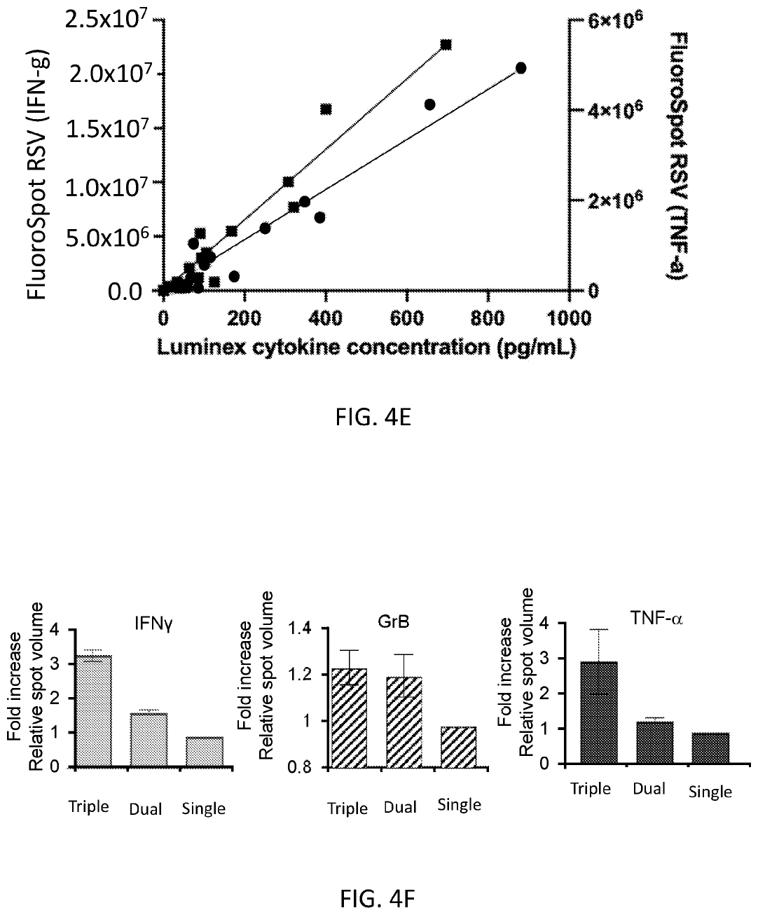

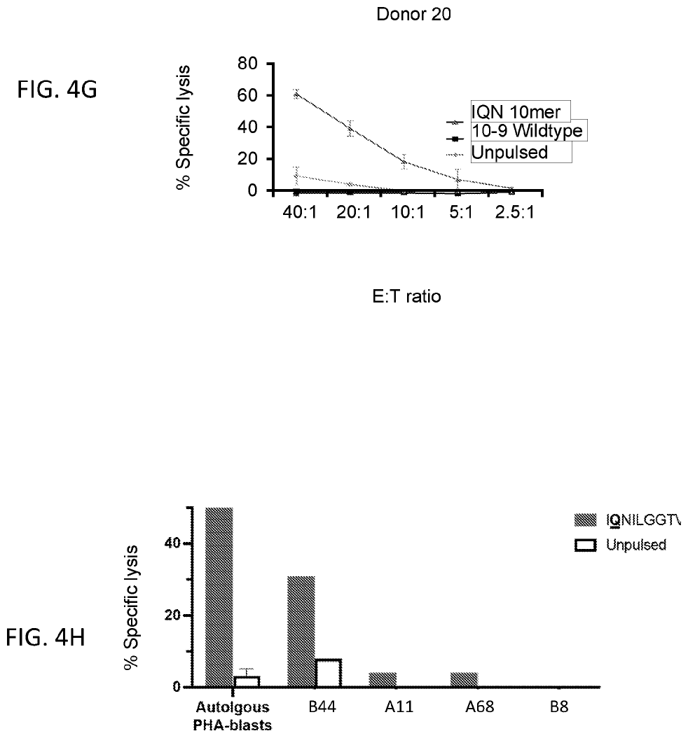

It was next sought to comprehensively characterize the functional profile of mutant-specific neo^{IDH2 R140Q}-T cells. First, the polyfunctionality was assessed of these cells using a FluoroSpot assay designed to simultaneously detect IFN-γ. TNF-α and granzyme B (GrB). The inventors initially examined each of these analytes individually (FIG. 4A, representative donor; right, n=6), and then in combination to assess polyfunctionality (i.e., capable of producing 2 or more effector molecules following antigen exposure). FIG. 4B shows a representative FluoroSpot region from donor 20 (reactive against the IQN-10mer), which contained cells capable of triple (designated by * symbol) or dual (IFN-γ+TNF-α, “v” symbol; IFN-γ+GrB, arrow symbol; TNF-α+GrB. “o” symbol) effector molecules. Of all the IFN-γ secreting cells in this donor, approximately 50% were polyfunctional: 39% additionally produced TNF-α (31%) or GrB (7%), while 12% secreted all three effector molecules (triple secretors) (FIG. 4C, left). Similarly, 12% of all TNF-α secreting cells also produced triple effector molecules (FIG. 4C, right), attesting to the Th1-polarized and polyfunctional nature of neo^{IDH2 R140Q}-reactive T cells.

A major feature of the FluoroSpot assay is the ability to detect not only the frequency of reactive cells (represented by the number of SFCs), but also provide a quantitative assessment of effector molecules based on the relative amount of a given analyte secreted per reactive cell, using a surrogate measure called the Relative Spot Volume (RSV). The RSV is derived from the size and intensity of each fluorescence signal-such that colonies with higher RSVs are visible as larger and brighter spots (FIG. 4D), corresponding to a greater volume of cytokines released and captured on the FluoroSpot plate. To validate this method of quantitation, we stimulated neo-reactive T cells with immunogenic peptides and compared the FluoroSpot-derived RSV values with traditional ELISA-based quantitation and saw a strong linear correlation between the two assays (FIG. 4E). Consequently, the single, dual and triple secretors were further profiled by comparing their capacity to secrete effector molecules upon antigen exposure based on the RSV. Triple secretors released approximately 3 fold more IFN-γ and TNF-«, and 1.2 fold more GrB than single secretors (FIG. 4E, n=4). This indicates that not only do polyfunctional neo^{IDH R140Q} T cells produce multiple effector molecules, but on a per cell basis produce more that their single-producing counterparts.

Lastly, to examine their cytotoxic potential, neo^{IDH R140Q} T cells were incubated with peptide-pulsed PHA blasts or PHA blasts alone as the negative control. FIG. 4F shows data from donor 20 with strong activity to the mutant-specific epitope IQNILGGTVF (SEQ ID NO:1) (SFC 412/2×10⁵ cells), which specifically lysed autologous mutant peptide-pulsed PHA blasts (60% specific lysis, E: T 40:1), while showing no activity against wildtype-pulsed or unpulsed PHA blasts. These results were confirmed for donor 13 and donor 5 with specificities towards TIQNILGGTV (SEQ ID NO:2) and IQNILGGTV (SEQ ID NO:3), respectively (E: T 40:1, FIG. 4G), demonstrating that neo^{IDH R140Q} T cells can mediate mutant-specific cytolytic effects in vitro. The cytotoxicity assay was additionally used to identify the HLA restricting element for each of these peptides using autologous and a panel of partially HLA-matched PHA blasts as targets. For donor 20 (A*11.68; B*8.44) for example, reactive T cells recognised autologous and HLA-B44 matched targets, thus confirming that neo-T cell activity against IQNILGGTV (SEQ ID NO:3) was restricted to HLA-B44 (FIG. 4H). Table 5 shows the summary results for all CD8+ epitopes identified.

TABLE 5
Examples of CD8+ Epitopes and Corresponding
HLA-Restriction

| CD8+ Epitope | HLA-restriction |
|---|---|
| IQNILGGTVF | B44 |
| IQNILGGTV | B15 |
| TIQNILGGTV | To be determined |

(epitopes are represented by SEQ ID NO: 1, SEQ ID NO: 3, and SEQ ID NO: 2 from top to bottom)

### Example 2

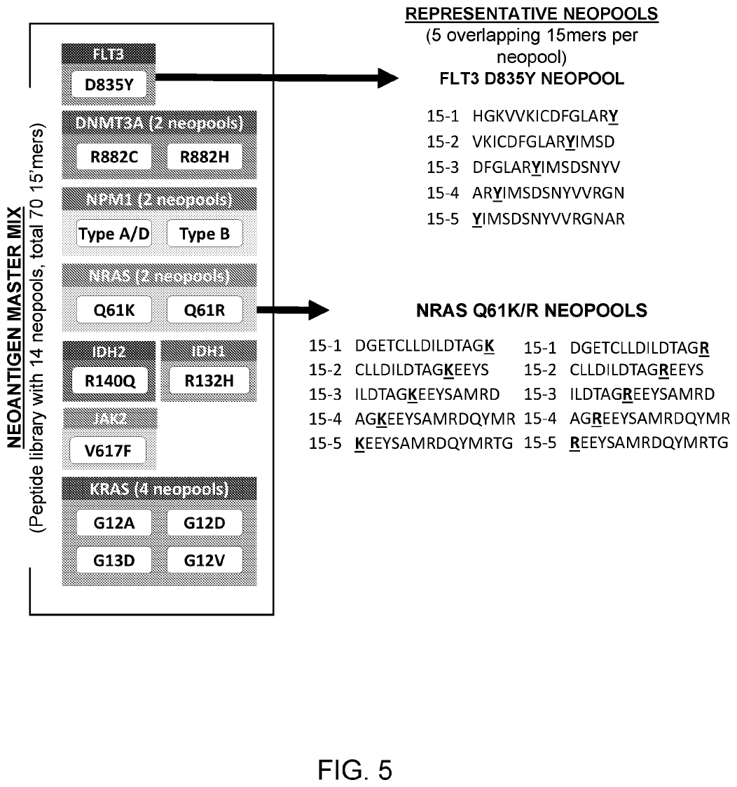

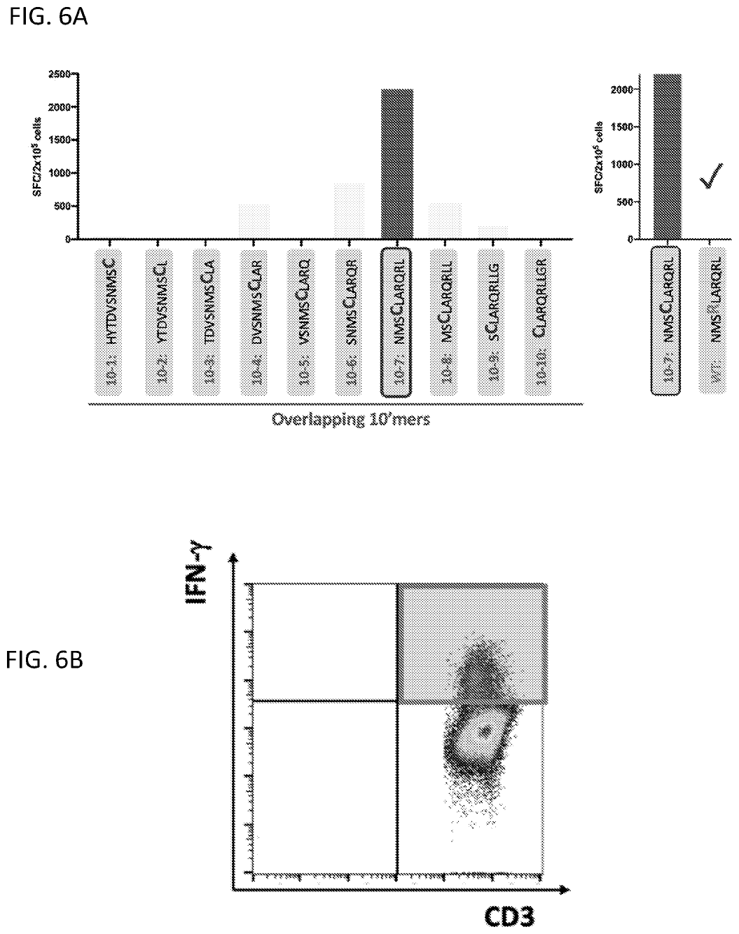

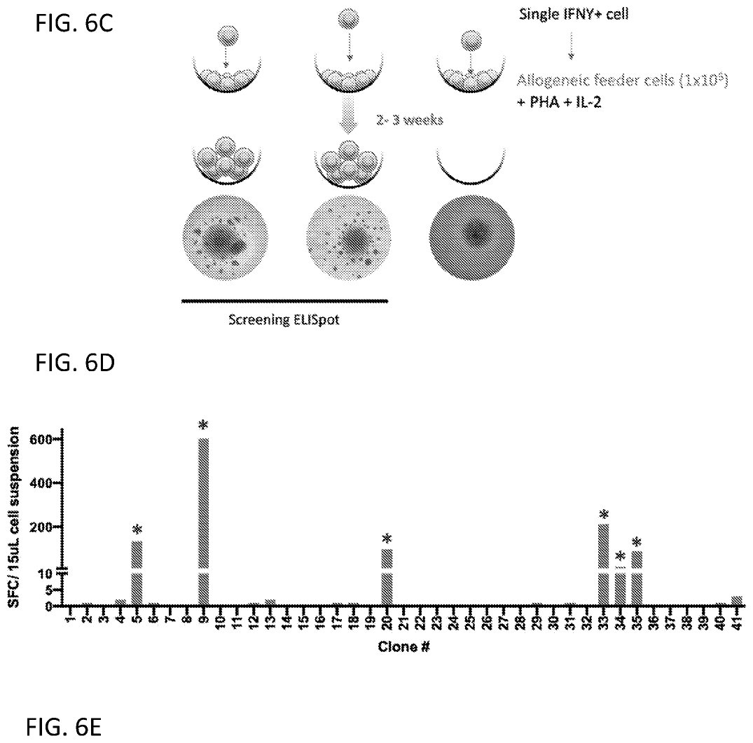

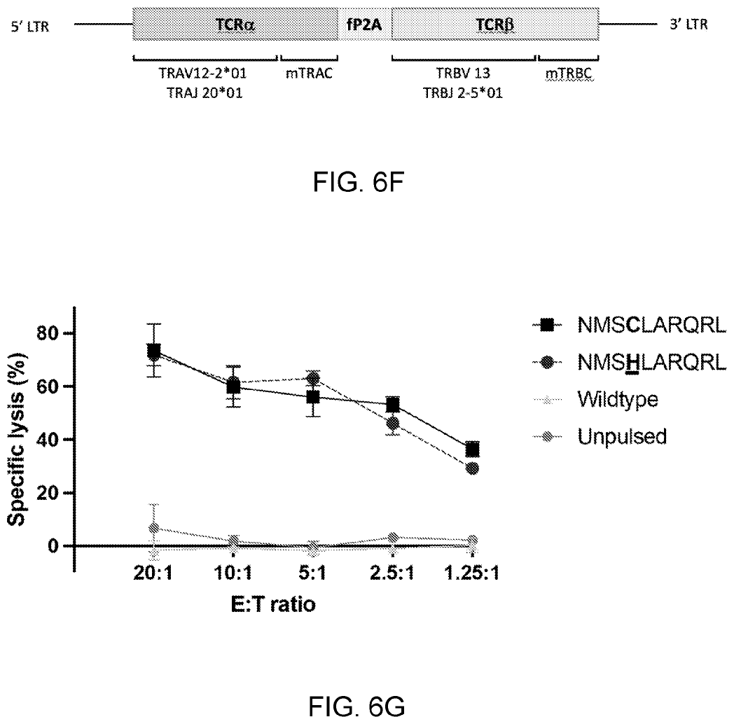

Neospecific T cells generated from Donor 8 were specific towards the DNMT3A R882C mutation, and the reactivity was mediated by CD8 T cells (ICS data, not shown). To identify the minimal epitope, the neoantigen-specific T cells were exposed to a panel of DNMT3A R882C 10mers overlapping by 9AA. After an overnight incubation, IFN-γ ELISpot was performed that identified the 10-7 NMS peptide as the immunogenic epitope (FIG. 6A, left), with no activity towards the analogous wildtype sequence (right). Next, to isolate T cell clones for TCR sequencing, the inventors stimulated the neoantigen-specific T cells for 6 hours with the 10-7 NMS peptide, labeled the IFNγ+ population, and selected them using a magnetic column (FIG. 6B). Using limiting dilution techniques, one IFN-γ positive cell was seeded in each well of a 96 well plate, with allogeneic feeders in presence of IL2 (FIG. 6C) to support expansion. After 2-3 weeks in culture, 41 colonies showed growth, of which 6 colonies remained specific for the 10-7 NMS peptide (FIG. 6D). These colonies were then sequenced, which identified the V (D) J and CDR3 regions (FIG. 6E) of the DNMT3A-R882C neo-specific TCR. The neo-TCR construct was designed, utilizing the sequenced α and β V (D) J regions linked to the murine constant region to facilitate α- and β-chain binding (FIG. 6F). This was then cloned into the SFG retroviral backbone and used to transduce PBMCs. As shown in FIG. 6G, cells transduced with the neo-TCR were able to lyse autologous peptide pulsed targets for both the R882C and R882H mutation, with no response towards unpulsed or wildtype pulsed targets—thus demonstrating the dual specificity and function of the DNMT3A-R882C/R882H TCR.

What is claimed:

1. A method of generating transgenic neoantigen-specific T cell receptor (TCR)-based immunotherapy that target at least one peptide comprising part or all of a neoantigen present in a hematological malignancy or pre-cancerous state, said method comprising the steps of:
(a) contacting a population of antigen presenting cells (APCs) with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, thereby producing pepmix-loaded APCs;
contacting peripheral blood mononuclear cells (PBMCs) from an individual with the pepmix-loaded APCs and performing at least one in vitro stimulation to produce a population of antigen-specific T cells that are capable of responding to at least one of the peptides; or
(b) contacting PBMCs from an individual with cancer or a healthy individual with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, and
performing at least one in vitro stimulation to produce a population of antigen-specific T cells that are capable of responding to at least one of the peptides;
wherein in (a) or (b) the in vitro stimulation comprises culturing in a medium supplemented with two or more cytokines selected from the group consisting of IL-7, IL-12, IL-15 and IL-6 to produce the hematological neoantigen-specific T cells; and
(c) generating the TCR-modified immunotherapy from the antigen binding region of the TCRs or the entire TCRs of the hematological neoantigen-specific T cells.

2. The method of claim 1, wherein the TCR-modified immunotherapy comprises TCR-modified immune cells, a TCR-drug conjugate, TCR-radiotherapy conjugate, TCR-linked CD3 complex, or a mixture thereof.

3. The method of claim 1 or 2, further comprising the step of assaying for neo-epitope specificity of the neoantigen-specific T cells by assessing T cell activity against one or more particular peptides comprising the mutated neoantigen sequence in comparison to T cell activity against a correlative peptide comprising the corresponding wild type sequence.

4. The method of claim 3, wherein the T cell activity is assessed by production of IFNγ, TNFα, and/or Granzyme B and/or by direct cytotoxicity of neopeptide-expressing targets.

5. The method of claim 3 or 4, wherein the T cell activity is assessed by ELIspot, Flurospot, single cell RNA sequencing, cytotoxicity assay, and/or one or more intracellular cytokine assays.

6. The method of any one of claims 1-5, further comprising the step of one or more additional in vitro stimulation steps.

7. The method of claim 6, wherein the one or more additional in vitro stimulation steps comprises culturing the cells in a medium comprising IL-7 and one or both of IL-15 and IL-2.

8. The method of any one of claims 1-7, wherein the stimulation step occurs in a multiwell substrate or flask or a vessel with a gas permeable membrane.

9. The method of any one of claims 1-8, wherein the APCs are dendritic cells, allogeneic feeder cells, cells from lymphblastoid cell lines, activated T cells, PHA blasts with irradiation, PHA blasts without irradiation, B cells, monocytes, genetically modified mesenchymal stem cells, tumor cell lines, cells from K562 cell line modified to express one or more costimulatory molecules, a combination of stimulator cells that either present antigens and/or provide costimulation and/or produce one or more soluble factors that promote enrichment of the cells in vitro, or a mixture thereof.

10. The method of any one of claims 1-9, wherein the mutated neoantigen sequence comprises one or more modified amino acids.

11. The method of claim 10, wherein the one or more modified amino acids comprises one or more amino acid substitutions, one or more amino acid deletions, one or more insertions, one or more inversions, or one or more translocations.

12. The method of any one of claims 1-11, wherein the PBMCs are from an individual that has cancer or a precancerous condition harboring one or more of the neoantigens.

13. The method of any one of claims 1-11, wherein the PBMCs are from an individual that is healthy and does not have cancer or a precancerous condition.

14. The method of any one of claims 1-13, further comprising the step of identifying the α and/or β T-cell receptor (TCR) polypeptide sequences of the neoantigen-specific T cells.

15. The method of claim 14, wherein the identifying step comprises single cell sequencing methods or bulk sequencing methods.

16. The method of claim 14, wherein specific neoantigen-reactive cells are captured using flow cytometry or magnetic selection for sequencing.

17. The method of claim 15 or 16, wherein specific cells are determined by IFNγ-secretion, TNFα secretion, or expression of activation markers such as CD137, CD28, and/or CD69.

18. The method of any one of claims 14-17, further comprising the step of cloning the α and/or β T-cell receptor (TCR) sequences into a vector, wherein the α and/or β TCR sequences encompass the antigen binding domains or the entire TCR sequences.

19. The method of claim 18, wherein the vector is a viral vector or a non-viral vector.

20. The method of claim 19, wherein the viral vector is retroviral, lentiviral, adenoviral, or adeno-associated viral vector.

21. The method of claim 19, wherein the non-viral vector is a plasmid or transposon or wherein the sequences are incorporated into the genome of the cell.

22. The method of any one of claims 18-21, further comprising the step of engineering immune cells to express an engineered TCR comprising the α and/or β T-cell receptor (TCR) sequences, thereby producing engineered hematological neoantigen-specific immune cells.

23. The method of claim 22, wherein the immune cells are T cells, antigen-specific cells, activated T cells, memory cells, naïve T cells, macrophages, B cells, natural killer cells, natural killer T cells, or a mixture thereof.

24. The method of claim 22 or 23, wherein the human constant region is replaced with a constant region that is not human.

25. The method of any one of claims 22-24, wherein the engineered TCR comprises sequences or modifications that stabilize the α and β chains of the engineered TCR to facilitate suitable transgenic TCR pairing.

26. The method of claim 25, wherein the engineered TCR comprises a murine constant region, has swapped constant domorins of α and β chains, comprises γδ constant domains, comprises CD3zeta, comprises a single chain TCR format, or wherein the cells comprise disruption of one or more molecules of the endogenous TCR complex.

27. The method of claim 26, wherein the disruption of one or more molecules of the endogenous TCR complex is by use of siRNA, CRISPR, TALEN, or ZFN.

28. The method of any one of claims 1-27, wherein the hematological neoantigen is associated with myeloid malignancies at the malignant and/or pre-malignant phase.

29. The method of claim 28, wherein the neoantigen comprises a mutation within the KRAS, NRAS, p53, BRAF, EZH2, MYD88 (NF-κB), PAX5, DNMT3A, NPM1, IDH1, IDH2, JAK2, CALR, FLT3, KMT3A, TET2, ASXL1, CEBPA, RUNX1, PTPN11, SRSF2, MLL, KIT, EZH2, SF3B1, CBL, U2AF1, BCOR, GATA2, MYC, NOTCH1, NOTCH2, CARD11, CD79A/B, CXCR4, BIRC3, TRAF3, TCF3, KLF2, PLGG1, STAT3, STAT5B, PRKCB, or ALKgene.

30. The method of any one of claims 1-27, wherein the hematological neoantigen is associated with lymphoid malignancies.

31. The method of claim 30, wherein neoantigen comprises a mutation within the BCR-ABL1, ETV6-RUNX1, or TCF3-PBX1 gene.

32. The method of any one of claims 1-31, wherein the neoantigen is from any one of Tables 1-3.

33. The method of any one of claims 14-31, wherein the α chain comprises SEQ ID NO:49 or SEQ ID NO:51.

34. The method of any one of claims 14-31, wherein the β chain comprises SEQ ID NO:50 or SEQ ID NO:52.

35. Hematological neoantigen-specific T cells produced by the method of any one of claims 1-21.

36. The cells of claim 35, comprised in a pharmaceutically acceptable excipient.

37. Engineered hematological neoantigen-specific immune cells produced by the method of any one of claims 22-34.

38. The cells of claim 37, comprised in a pharmaceutically acceptable excipient.

39. The cells of claim 37 or 38, wherein the TCR is modified to increase TCR affinity and/or avidity and/or functional avidity to target the neoantigen.

40. The cells of claim 39, wherein the TCR is modified by substitution of one or more amino acids in the complementarity-determining region.

41. The cells of any one of claims 37-40, wherein the cells comprise more than one engineered TCR.

42. The cells of claim 41, wherein two or more engineered TCRs target different neoantigens, tumor antigens, or viral antigens.

43. The cells of any one of claims 37-42, wherein the cells comprise one or more heterologous molecules other than the engineered TCR and/or wherein the cells comprise disruption of one or more endogenous genes to the cells.

44. The cells of claim 43, wherein the heterologous molecule is a transgenic receptor; a modulator that enhances persistence and/or function of the cells; provides anticancer activity, or a combination thereof.

45. The cells of claim 44, wherein the transgenic receptor is a chimeric antigen receptor, a TCR targeting a non-neoantigen tumor-associated antigen, a cytokine receptor, or a chimeric cytokine receptor.

46. The cells of claim 44, wherein the modulator is a cytokine or cytokine receptor, chemokine receptor, or chemokine.

47. The cells of any one of claims 44-46, wherein the heterologous molecule provides an additive or synergistic antitumor effect compared to the antitumor effect of the cells in the absence of the heterologous molecule.

48. The cells of any one of claims 37-47, wherein the cells are further defined as being specific for another antigen than the neoantigen.

49. The cells of claim 48, wherein the antigen other than the neoantigen is a germline tumor antigen, a viral antigen, or wherein the cells are alloreactive T cells.

50. The cells of any one of claims 37-49, comprised in one or more cryoprotectants.

51. The cells of any one of claims 37-50, wherein said cells are housed in a depository.

52. The cells of any one of claims 37-50, wherein said cells comprise a safety switch or suicide gene.

53. The cells of claim 52, wherein the suicide gene is inducible iCas9.

54. The cells of claim 52, wherein the safety switch comprises CD20 or truncated EGFR.

55. A method of treating an individual with hematological cancer or a hematological precancerous condition, comprising the step of administering to the individual a therapeutically effective amount of the hematological neoantigen-specific T cells of claim 35 or 36, or administering to the individual a therapeutically effective amount of any one of the engineered hematological neoantigen-specific immune cells of claims 37-52.

56. The method of claim 55, wherein the cells are administered by injection.

57. The method of claim 56, wherein the injection is intravenous.

58. The method of any one of claims 55-57, wherein the PBMCs from which the engineered hematological neoantigen-specific immune cells were produced are from the individual.

59. The method of any one of claims 55-57, wherein the PBMCs from which the engineered hematological neoantigen-specific immune cells were produced are not from the individual.

60. A method of preventing or reducing the risk for an individual for development of hematological cancer from a precancerous condition, comprising the step of administering to the individual a therapeutically effective amount of the hematological neoantigen-specific T cells of claim 35 or 36, or administering to the individual a therapeutically effective amount of any one of the engineered hematological neoantigen-specific immune cells of claims 37-49.

61. The method of claim 60, wherein the cells are administered by injection.

62. The method of claim 61, wherein the injection is intravenous.

63. The method of any one of claims 60-62, wherein the PBMCs from which the engineered hematological neoantigen-specific immune cells were produced are from the individual.

64. The method of any one of claims 60-63, wherein the PBMCs from which the engineered hematological neoantigen-specific immune cells were produced are not from the individual.

65. A composition, comprising the identified α and/or β T-cell receptor (TCR) polypeptides of any one of claims 14-34.

66. The composition of claim 65, wherein the α and/or β T-cell receptor (TCR) polypeptides are labeled, or wherein another molecule in the composition is labeled.

67. The composition of claim 66, wherein the label is fluorescent, colorimetric, photochromic, radioactive, is an Fc fragment of a human or non human immunoglobulin, or is an enzyme that is activateable or deactivatiable upon contact with the neoantigen.

68. The composition of claim 65, wherein said α and/or β T-cell receptor (TCR) polypeptides are comprised in a transgenic molecule.

69. The composition of claim 68, wherein the transgenic molecule is a chimeric antigen receptor, a multi-specific T cell engager, or wherein the α and/or β T-cell receptor (TCR) polypeptides are conjugated to a drug and/or radiotherapeutic molecule.

70. The composition of claim 69, wherein the drug is a hematological chemotherapeutic.

71. A method of detecting a hematological neoantigen in a sample from an individual, comprising the step of contacting an effective amount of the composition of any one of claims 65-67 with the sample.

72. The method of claim 71, wherein the sample is a research sample or a clinical sample.

73. A method of diagnosing a hematological cancer or precancerous condition in an individual, comprising the step of contacting an effective amount of the composition of any one of claims 65-67 with the sample.

74. The method of claim 73, wherein the sample comprises peripheral blood.

75. The method of claim 73 or 74, wherein the sample comprises memory cells that are specific for the neoantigen.

76. The method of any one of claims 73-75, wherein the individual is suspected to have cancer.

77. The method of any one of claims 73-75, wherein the individual is at risk for cancer compared to the general population.

78. The method of any one of claims 73-77, wherein when neoantigen-specific memory cells are identified from the sample, the individual is given an effective amount of one or more cancer therapies.

79. The method of claim 78, wherein the cancer therapy comprises immunotherapy directed to the neoantigen.

80. The method of claim 79, wherein the immunotherapy comprises engineered T cells that express the antigen binding domain or all of the TCR.

81. The method of claim 79 or 80, wherein the immunotherapy comprises the antigen binding domain or all of the TCR linked to an antibody or small molecule.

82. The method of any one of claims 71-81, wherein the contacting utilizes the antigen binding domain or all of the TCR that is labeled with a fluorescent label, colorimetric label, photochromic label, radioactive label, an Fc fragment of a human or non human immunoglobulin, or with an enzyme that is activateable or deactivatiable upon contact with the neoantigen.

83. A method of activating or costimulating immune cells, comprising the step of contacting immune effector cells with an effective amount of the composition of any one of claims 65-70.

84. The method of claim 70, wherein the activating comprises cytokine signaling.

85. The method of claim 83 or 84, wherein the cell is further defined as comprising a molecule that provides costimulation when in contact with a T cell.

86. The method of claim 85, wherein the molecule is CD80 or CD86.

87. An immunological composition, comprising a neopeptide comprising a neoantigen from KRAS, NRAS, p53, BRAF, EZH2, MYD88 (NF-κB), PAX5, DNMT3A, NPM1, IDH1, IDH2, JAK2, CALR, FLT3, KMT3A, TET2, ASXL1, CEBPA, RUNX1, PTPN11, SRSF2, MLL, KIT, EZH2, SF3B1, CBL, U2AF1, BCOR, GATA2, MYC, NOTCH1, NOTCH2, CARD11, CD79A/B, CXCR4, BIRC3, TRAF3, TCF3, KLF2, PLGG1, STAT3, STAT5B, PRKCB, or ALK or wherein the neoantigen is from a translocation from ETV6-RUNX1, CBFb/YH11, BCR-ABL1, ETV6-RUNX1, IGH-IL3, TCF3-PBX1, NUP213-ABL1, PICALM-MLLT10; those involving of MLL, or TCR locus.

88. A method of identifying an immunological composition for cancer or a precancerous condition associated with one or more neoantigens, comprising the step of assaying a plurality of peptides encompassing one or more neoantigens for immunogenicity to identify one or more peptides that elicit a T cell response from multiple donors.

89. The method of claim 88, further comprising the step of formulating the identified peptide(s) with an adjuvant and/or in a pharmaceutically acceptable excipient.

90. The method of claim 88 or 89, further comprising the step of administering a therapeutically effective amount of the formulated peptide(s) to an individual with cancer or a precancerous condition.

91. An immunological composition produced by the method of any one of claims 88-90.
